SEQUENCE LISTING (3 sequences)

SEQ ID NO: 1 - protein, 11 aa, Homo sapiens
TLNSVLNSNAI

SEQ ID NO: 2 - protein, 11 aa, Homo sapiens
SVLNSNAIKNL

SEQ ID NO: 3 - protein, 10 aa, Homo sapiens
SKMYHTKGQE

(19) United States
(12) Patent Application Publication
(10) Pub. No.: US 2003/0165501 A1
(43) Pub. Date: Sep. 4, 2003

(54) Treatment involving Dkk-1 or antagonists thereof

(75) Inventors: Venita I. DeAlmeida, San Carlos, CA (US); Timothy A. Stewart, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/077,065

(22) Filed: Feb. 15, 2002

Related U.S. Application Data
(60) Provisional application No. 60/269,435, filed on Feb. 16, 2001.

(51) Int. Cl. A61K 39/395

(52) U.S. Cl. 424/145.1

(57) ABSTRACT

Antagonists to Dickkopf-1 (Dkk-1) protein are administered in effective amounts to treat disorders involving insulin resistance, such as non-insulin-dependent diabetes mellitus (NIDDM), hypoinsulinemia, and disorders involving muscle atrophy, trauma, or degeneration. Preferably, the antagonists are composed of compositions comprising antibodies directed to Dkk-1 in a pharmaceutically acceptable carrier for use in blocking the effects of Dkk-1. Additionally provided is a method of treating obesity or hyperinsulinemia in a mammal by administering an effective amount of Dkk-1 to a mammal. Also provided are methods of diagnosing insulin resistance, hyper- and hypoinsulinemia, obesity, and related disorders using Dkk-1 as a target and non-human transgenic animals that overexpress dkk-1 nucleic acid.

### RELATED APPLICATIONS

This application is a non-provisional application filed under 37 CFR 1.53(b)(1), claiming priority under 35 USC 119(e) to provisional application number 60\269,435 filed Feb. 16, 2001, the contents of which are incorporated herein by reference.

### BACKGROUND OF THE INVENTION

1. Field of the Invention

The present invention provides for the diagnosis and treatment of disorders involving obesity, insulin resistance, hypoinsulinemia, and hyperinsulinemia and for repairing and regenerating muscle in mammals. More particularly, the present invention relates to the use of Dickkopf-1 (Dkk-1) protein to treat obesity and hyperinsulinemia and to the use of antagonists that bind to Dkk-1 and/or neutralize its activity in the treatment of insulin resistance and hypoinsulinemia, and in muscle repair.

2. Description of Related Disclosures

The Dickkopf (dkk) proteins are a group of secreted proteins that modulate Wnt activity (Krupnik et al., Gene, 238: 301-313 (1999); Monaghan et al., Mech. Dev., 87: 45-56 (1999); Roessler et al., Cell Genet., 89: 220-224 (2000)). This family is composed of four members, which are highly related and contain two conserved cysteine-rich domains (WO 00/52047 published Sep. 8, 2000).

Dkk-1 (WO 99/46281 published Sep. 16, 1999, wherein the Dkk-1 is designated as PRO1008 and is encoded by DNA57530; WO 00/18914 published Apr. 6, 2000; WO 00/52047 published Sep. 8, 2000; WO 98/46755 published Oct. 22, 1998) was first identified as an inducer of head formation in Xenopus by inhibition of Wnt signaling (Glinka et al., Nature, 391: 357-362 (1998)), and subsequently shown to be involved in limb development (Grotewold et al., Mech. Dev. 89: 151-153 (1999)) and inhibitory to Wnt-induced morphological transformation (Fedi et al., J. Biol. Chem., 274: 19465-19472 (1999)).

Recent studies indicate that the Dkks act by binding to the low-density lipoprotein-related protein, LRP6, which acts as a co-receptor for Wnt signaling (Mao et al., Mol. Cell. 7: 801-809 (2001); Pinson et al., Nature, 407: 535-538 (2000); Tamai et al., Nature, 407: 530-535 (2000); Wehrli et al., Nature, 407: 527-530 (2000)). Dkk-1 antagonizes Wnt signaling by binding to LRP6 at domains distinct from those involved in its interaction with Wnt and Frizzled, thus inhibiting LRP6-mediated Wnt/β-catenin signaling (Bafico et al., Nat. Cell. Biol., 3: 683-686 (2001); Mao et al., Nature, 411: 321-325 (2001); Semenov et al., Current Biology 11: 951-961 (2001)).

Proteins of the Wnt family play a key role in embryonic development and differentiation of various cell types (Peifer and Polakis, Science, 287: 1606-1609 (2000)). The Wnt signaling pathway is activated by the interaction between secreted Wnts and their receptors, the frizzled proteins (Hlsken and Behrens, J. Cell. Sci., 113: 3545-3546 (2000)), with the LDL receptor-related proteins LRP5 and LRP6 acting as co-receptors (Mao et al., Mol. Cell., supra; Pinson et al., supra; Tamai et al., supra; Wehrli et al., supra). The downstream effects of Wnt signaling include activation of Disheveled (Dvl1) protein, resulting in the activation and subsequent recruitment of Akt to the Axin-β-catenin-GSK3β-APC complex (Fukumoto et al., J. Biol. Chem., 276: 17479-17483 (2001)). This is followed by the phosphorylation and inactivation of GSK3β, resulting in inhibition of phosphorylation and degradation of β-catenin. The accumulated β-catenin is translocated to the nucleus where it interacts with transcription factors of the lymphoid enhancer factor-T cell factor (LEF/TCF) family and induces the transcription of target genes.

Two of the downstream effectors of Wnt signaling, Akt and GSK3β, are key intermediates in the insulin signaling pathway/glucose metabolism. Wnt signaling is involved in the regulation of muscle differentiation (Borello et al., Development, 126: 4247-4255 (1999); Cook et al., Embo. J., 15: 4526-4536 (1996); Cossu and Borello, Embo. J., 18: 6867-6872 (1999); Ridgeway et al., J. Biol. Chem., 275: 32398-32405 (2000); Tian et al., Development, 126: 3371-3380 (1999); Toyofuku et al., J. Cell. Biol., 150: 225-241 (2000)) and adipogenesis (Ross et al., Science, 289: 950-953 (2000)), and inhibition of Wnt signaling can stimulate the trans-differentiation of myocytes to adipocytes (Ross et al., supra).

Treatment with Wnt/Wg-conditioned medium for short time periods did not result in Akt activation and GSK3β phosphorylation at Ser9, although free β-catenin was accumulated in the cytosol (Ding et al, J. Biol. Chem., 275: 32475-32481 (2000). In contrast, prolonged or constitutive Wnt stimulation resulted in Akt activation and involvement in Wnt signaling (Fukumoto et al., supra). In HepG2 cells insulin signaling stimulates β-catenin, an intermediate of Wnt signaling, through two signaling pathways: activation of P13-kinase and Akt resulting in GSK3b inhibition and through Ras activation (Desbois-Mouthon et al., Oncogene, 20: 252-259 (2001)). However, in 293, C57, and CHOIR cells, insulin did not affect β-catenin cytosolic levels, and more significantly, neither the phosphorylation status of Ser9 of GSK3 β nor the activity of protein kinase B was regulated by Wnt (Ding et al., supra).

Insulin resistance is a condition where the presence of insulin produces a subnormal biological response. In clinical terms, insulin resistance is present when normal or elevated blood glucose levels persist in the face of normal or elevated levels of insulin. It represents, in essence, a glycogen synthesis inhibition, by which either basal or insulin-stimulated glycogen synthesis, or both, are reduced below normal levels. Insulin resistance plays a major role in Type 2 diabetes, as demonstrated by the fact that the hyperglycemia present in Type 2 diabetes can sometimes be reversed by diet or weight loss sufficient, apparently, to restore the sensitivity of peripheral tissues to insulin.

It is now appreciated that insulin resistance is usually the result of a defect in the insulin receptor signaling system, at a site post binding of insulin to the receptor. Accumulated scientific evidence demonstrating insulin resistance in the major tissues that respond to insulin (muscle, liver, adipose), strongly suggests that a defect in insulin signal transduction resides at an early step in this cascade, specifically at the insulin receptor kinase activity, which appears to be diminished (Haring, Diabetalogia, 34: 848 (1991)).

Several studies on glucose transport systems as potential sites for such a post-receptor defect have demonstrated that both the quantity and function of the insulin-sensitive glucose transporter (GLUT4) is deficient in insulin-resistant states of rodents and humans (Garvey et al., Science, 245: 60 (1989); Sivitz et al., Nature, 340: 72 (1989); Berger et al., Nature, 340: 70 (1989); Kahn et al., J. Clin. Invest., 84: 404 (1989); Charron et al., J. Biol. Chem., 265: 7994 (1990); Dohm et al., Am. J. Physiol., 260: E459 (1991); Sinha et al., Diabetes, 40: 472 (1991); Friedman et al., J. Clin. Invest., 89: 701 (1992)). A lack of a normal pool of insulin-sensitive glucose transporters could theoretically render an individual insulin resistant (Olefsky et al., in Diabetes Mellitus, Rifkin and Porte, Jr., Eds. (Elsevier Science Publishing Co., Inc., New York, ed. 4, 1990), pp. 121-153). However, some studies have failed to show downregulation of GLUT4 in human NIDDM, especially in muscle, the major site of glucose disposal (Bell, Diabetes, 40: 413 (1990); Pederson et al., Diabetes, 39: 865 (1990); Handberg et al, Diabetologia, 33: 625 (1990); Garvey et al., Diabetes, 41: 465 (1992)).

Evidence from in vivo studies in animal models and clinical studies indicate that insulin resistance in Type 2 diabetes can result from alterations in expression and activity of intermediates in the insulin signal transduction pathway, from alteration in the rate of insulin-stimulated glucose transport or from alterations in translocation of GLUT4 to the plasma membrane (Zierath et al., Diabetologia, 43: 821-835 (2000)). Evidence from animal studies suggests that insulin-signaling defects in muscle alter whole-body glucose homeostasis (Saad et al., J. Clin. Invest., 90: 1839-1849 (1992); Folli et al., J. Clin. Invest., 92: 1787-1794 (1993); Heydrick et al., J. Clin. Invest., 91: 1358-1366 (1993); Saad et al., J. Cin. Invest., 92: 2065-2072 (1993); Heydrick et al., Am. J. Physiol., 268: E604-612 (1995)) and defects in intermediates in the insulin-signaling cascade including the IR, IRS-1, and PI 3-kinase can lead to reduced glucose transport and reduced insulin-stimulated GLUT4 translocation in skeletal muscle from insulin-resistant and Type 2 diabetic subjects.

In some examples, altered expression of IRS-1 (Saad et al., 1992, supra; Saad et al., 1993, supra; Goodyear et al., J. Clin. Invest., 95: 2195-2204 (1995)), PI 3-kinase (Anai et al., Diabetes, 47: 13-23 (1998)), and GSK-3 (Nikoulina et al., Diabetes, 49: 263-271 (2000)), and decreased levels of PKCθ (Chalfant et al., Endocrinology, 141: 2773-2778 (2000)) and PTP1B (Dadke et al., Biochem. Biophys. Res. Commun., 274: 583-589 (2000)) have been observed. Decreased phosphorylation of IR (Amer et al., Diabetologia, 30: 437-440 (1987); Maegawa et al., Diabetes, 44: 815-819 (1991); Saad et al., 1992, supra, Saad et al., 1993, supra, Goodyear et al., supra), IRS-1 (Saad et al., 1992, supra; Saad et al., 1993, supra; Goodyear et al., supra), and Akt (Krook et al., Diabetes, 47: 1281-1286 (1998)) has also been observed in skeletal muscle of some Type 2 diabetic subjects.

Additionally, decreased activity of PI 3-kinase (Saad et al., 1992, supra; Heydrick et al., 1995, supra; Saad et al., 1993, supra; Goodyear et al., supra; Heydrick et al., 1993, supra; Folli et al., Acta Diabetol., 33: 185-192 (1996); Bjornholm etal., Diabetes, 46: 524-527 (1997); Andreelli et al., Diabetologia, 42: 358-364 (1999); Kim et al., J. Clin. Invest., 104: 733-741 (1999); Andreelli F, et al., Diabetologia, 43: 356-363 (2000); Krook et al., Diabetes, 49: 284-292 (2000)) and increased activity of GSK-3 (Eldar-Finkelman et al., Diabetes, 48: 1662-1666 (1999)), PKC (Avignon et al., Diabetes, 45: 1396-1404 (1996)), and PTP1B (Dadke et al., supra) have also been shown to be associated with Type 2 diabetes. Disruption of the p85 subunit of PI 3-kinase results in increased insulin sensitivity in mice (Terauchi et al., Nature Genetics, 21: 230-235 (1999)).

Additionally, the distribution of PKC isoforms is altered in skeletal muscle from diabetic animals (Schmitz-Peiffer et al., Diabetes, 46: 169-178 (1997)) and the content of PKCα, PKCβ, PKCε, and PKCδ is increased in membrane fractions and decreased in cytosolic fractions of soleus muscle in the non-obese Goto-Kakizaki (GK) diabetic rat (Avignon et al., supra).

Abnormal subcellular localisation of GLUT4 has been observed in skeletal muscle from insulin-resistant subjects with or without Type 2 diabetes (Vogt et al., Diabetologia, 35: 456-463 (1992); Garvey et al., J. Clin. Invest., 101: 2377-2386 (1998)), suggesting that defects in GLUT4 trafficking and translocation may cause insulin resistance in skeletal muscle. In vivo and in vitro studies have demonstrated a reduced rate of insulin-stimulated glucose transport in skeletal muscle in some Type 2 diabetic subjects (Andreasson et al, Acta Physiol. Scand., 142: 255-260 (1991); Zierath et al, Diabetologia, 37: 270-277 (1994); Bonadonna et al., Diabetes, 45: 915-925 (1996)).

It is noteworthy that, notwithstanding other avenues of treatment, insulin therapy remains the treatment of choice for many patients with Type 2 diabetes, especially those who have undergone primary diet failure and are not obese, or those who have undergone both primary diet failure and secondary oral hypoglycemic failure. But it is equally clear that insulin therapy must be combined with a continued effort at dietary control and lifestyle modification, and in no way can be thought of as a substitute for these. For achieving optimal results, insulin therapy should be followed with self-blood glucose monitoring and appropriate estimates of glycosylated blood proteins: Insulin may be administered in various regimens alone, two or multiple injections of short, intermediate or long-acting insulins, or mixtures of more than one type. The best regimen for any patient must be determined by a process of tailoring the insulin therapy to the individual patient's monitored response.

The current state of knowledge and practice with respect to the therapy of Type 2 diabetes is by no means satisfactory. The majority of patients undergo primary dietary failure with time. Although oral hypoglycemic agents are frequently successful in reducing the degree of glycemia in the event of primary dietary failure, many authorities doubt that the degree of glycemic control attained is sufficient to avoid the occurrence of the long-term complications of atheromatous disease, neuropathy, nephropathy, retinopathy, and peripheral vascular disease associated with longstanding Type 2 diabetes. The reason for this can be appreciated in the light of the realization that even minimal glucose intolerance, approximately equivalent to a fasting plasma glucose of 5.5 to 6.0 mmol/L, is associated with an increased risk of cardiovascular mortality (Fuller et al., Lancet, 1: 1373-1378 (1980)). It is also not clear that insulin therapy produces any improvement in long-term outcome over treatment with oral hypoglycemic agents.

Hyperinsulinemia is a condition where a higher-than-normal level of insulin is circulating within the body, whereas, conversely, hypoinsulinemia is a condition where a lower-than-normal level of insulin is circulating throughout the body. Hyperinsulinemia as a risk factor for restenosis after coronary balloon angioplasty (Imazu et al., Jpn Circ J., 65: 947-952 (2001)). Further, hyperinsulinemia is linked with hypertension (Imazu et al, Hypertens Res., 24: 531-536 (2001)). For example, hyperinsulinemia and hemostatic abnormalities are associated with silent lacunar cerebral infarcts in elderly hypertensive subjects, and hyperinsulinemia is a determinant of membrane fluidity of erythrocytes in essential hypertension (Kario et al., J. Am. Coll. Cardiol., 37: 871-877 (2001); Tsuda et al., Am. J. Hypertens., 14: 419-423 (2001)).

Obesity is a chronic disease that is highly prevalent in modern society and is associated not only with a social stigma, but also with decreased life span and numerous medical problems, including adverse psychological development, reproductive disorders such as polycystic ovarian disease, dermatological disorders such as infections, varicose veins, Acanthosis nigricans, and eczema, exercise intolerance, insulin resistance, hypertension, hypercholesterolemia, cholelithiasis, osteoarthritis, orthopedic injury, thromboembolic disease, cancer, and coronary heart disease. Rissanen et al., British Medical Journal, 301: 835-837 (1990). Treatment of obesity involves using appetite suppressors and other weight-loss inducers, dietary modifications, and the like, but, similar to the patients with insulin resistance, the majority of obese patients undergo primary dietary failure over time, thereby failing to achieve ideal body weight.

Thus, it can be appreciated that a superior method for treatment of both insulin resistance and obesity would be of great utility. Specifically, there is a need for effective agents that can be used in the diagnosis and therapy of individuals with insulin resistance, including NIDDM. In addition, considering the high prevalence of obesity in our society and the serious consequences associated therewith as discussed above, any therapeutic drug potentially useful in reducing the weight of obese persons could have a profound beneficial effect on their health. Finally, there is also a need for drugs to treat hyperinsulinemia, hypoinsulinemia, and muscle repair and regeneration.

### SUMMARY OF THE INVENTION

Accordingly, antagonists to Dkk-1, such as antibodies, are herein disclosed to be useful in the treatment of insulin resistance associated with, for example, glucose intolerance, diabetes mellitus, hypertension, and ischemic diseases of the large and small blood vessels and in the treatment of hypoinsulinemia. Further, Dkk-1 itself is disclosed herein as useful in reducing fat levels and in the treatment of hyperinsulinemia.

Specifically, the invention herein is the subject matter as claimed. It provides a method of treating insulin resistance or hypoinsulinemia in mammals comprising administering to a mammal in need thereof an effective amount of an antagonist to Dkk-1. Preferably, the mammal is human, the Dkk-1 is human Dkk-1, and/or the human has NIDDM. Also preferred is systemic administration. The antagonist is preferably an antibody that binds Dkk-1, and more preferably a monoclonal antibody that binds Dkk-1, and still more preferably one that neutralizes an insulin-resistance or hypoinsulinemic activity of Dkk-1. Most preferred is a monoclonal antibody prepared from a hybridoma having ATCC deposit no. PTA-3086, which is a neutralizing antibody. In a further preferred embodiment, another insulin-resistance-treating agent is administered in addition to the antagonist to treat the insulin-resistant disorder, or insulin is administered in addition to the antagonist to treat the hypoinsulinemia.

In another embodiment of the invention a method is provided for detecting the presence or onset of insulin resistance or hypoinsulinemia in a mammal. This method comprises the steps of:

(a) measuring the amount of Dkk-1 in a sample from said mammal; and

(b) comparing the amount determined in step (a) to an amount of Dkk-1 present in a standard sample, an increased level in the amount of Dkk-1 in step (a) being indicative of insulin resistance or hypoinsulinemia.

Preferably, the measuring is carried out using an anti-Dkk-1 antibody, such as a monoclonal antibody, in an immunoassay. Also, preferably such anti-Dkk-1 antibody comprises a label, more preferably a fluorescent label, a radioactive label, or an enzyme label, such as a bioluminescent label or a chemiluminescent label. Also, preferably, the immunoassay is a radioimmunoassay, an enzyme immunoassay, an enzyme-linked immunosorbent assay, a sandwich immunoassay, a precipitation assay, an immunoradioactive assay, a fluorescence immunoassay, a protein A immunoassay, or an immunoelectrophoresis assay. Also preferred is the method wherein the mammal is human, and human Dkk-1 is being measured. In a further preferred embodiment the insulin resistance is NIDDM.

In a further embodiment, the invention provides a kit for treating insulin resistance or hypoinsulinemia, said kit comprising:

(a) a container comprising an antagonist to Dkk-1, preferably an antibody that binds Dkk-1; and

(b) instructions for using the antagonist to treat insulin resistance or hypoinsulinemia.

In a preferred embodiment, the antibody is a monoclonal antibody, more preferably, one that neutralizes an insulin-resistance or hypoinsulinemic activity of Dkk-1. In another preferred embodiment, the kit further comprises a container comprising an insulin-resistance-treating agent or insulin, depending on the indication.

Additionally provided is a monoclonal antibody preparation prepared by hyperimmunizing mice with tagged Dkk-1 (preferably purified recombinant polyhistidine-tagged human Dkk-1) diluted in an adjuvant, fusing B-cells from the mice having anti-Dkk-1 antibody titers (preferably high titers) with mouse myeloma cells and obtaining supernatants, harvesting the supernatants, screening the harvested supernatants for antibody production, preferably by direct enzyme-linked immunosorbent assay, injecting positive clones showing the highest immunobinding after a second round of subcloning, preferably by limiting dilution, into primed mice for in vivo production of monoclonal antibodies, pooling ascites fluids from the mice, and purifying the ascites fluid pool, preferably by Protein A affinity chromatography, to produce the antibody preparation.

The invention further provides a hybridoma selected from the group consisting of ATCC Dep. No. PTA-3084, PTA-3085, PTA-3086, PTA-3087, PTA-3088, PTA-3089, and PTA-3097. The preferred hybridoma is ATCC Dep. No. PTA-3086. Also provided is an antibody prepared from one of the above hybridomas, preferably from PTA-3086.

The invention further provides a method of evaluating the effect of a candidate pharmaceutical drug on insulin resistance, hypoinsulinemia, or muscle repair comprising administering said drug to a non-human transgenic animal that overexpresses dkk-1 nucleic acid and determining the effect of the drug on glucose clearance from the blood of said animal, on circulating insulin levels in said animal, or on muscle differentiation, respectively. Preferably, the animal is a rodent, more preferably a mouse or rat, and most preferably a mouse. In another preferred embodiment, the dkk-1 nucleic acid overexpressed by the animal is under the control of a muscle-specific promoter, and the cDNA is overexpressed in muscle tissue.

In another embodiment, the invention provides a diagnostic kit for detecting the presence or onset of insulin resistance, hypoinsulinemia, hyperinsulinemia, or obesity, said kit comprising:

(a) a container comprising an antibody that binds Dkk-1;

(b) a container comprising a standard sample containing Dkk-1; and

(c) instructions for using the antibody and standard sample to detect insulin resistance, hypoinsulinemia, hyperinsulinemia, or obesity, wherein either the antibody that binds Dkk-1 is detectably labeled or the kit further comprises another container comprising a second antibody that is detectably labeled and binds to the Dkk-1 or to the antibody that binds Dkk-1. Preferably the anti-Dkk-1 antibody of the kit is a monoclonal antibody, more preferably one that neutralizes an insulin-resistance, hyperinsulinemic, hypoinsulinemic, or obesity activity of Dkk-1.

In another embodiment, the invention provides a method of treating obesity or hyperinsulinemia in mammals comprising administering to a mammal in need thereof an effective amount of Dkk-1. Preferably, the mammal is human and the Dkk-1 is human Dkk-1. Also preferably the administration is systemic. In another embodiment, the method further comprises administering an effective amount of a weight-loss agent.

In a further aspect, the invention provides a method for detecting the presence or onset of obesity or hyperinsulinemia in a mammal comprising the steps of:

(a) measuring the amount of Dkk-1 in a sample from said mammal; and

(b) comparing the amount determined in step (a) to an amount of Dkk-1 present in a standard sample, a decreased level in the amount of Dkk-1 in step (a) being indicative of obesity or hyperinsulinemia.

Preferably, the measuring is carried out using an anti-Dkk-1 antibody in an immunoassay.

Also, preferably the anti-Dkk-1 antibody comprises a label. The preferred labels and immunoassays are those as set forth above for the detection of the presence or onset of insulin resistance or hypoinsulinemia. In addition, in this method to detect obesity or hyperinsulinemia, the mammal is preferably human and human Dkk-1 is being measured.

In yet another embodiment, the invention provides a kit for treating obesity or hyperinsulinemia, said kit comprising:

(a) a container comprising Dkk-1; and

(b) instructions for using the Dkk-1 to treat obesity or hyperinsulinemia.

In a preferred embodiment the Dkk-1 is human Dkk-1 in the kit and it may further comprise a container with a weight-loss agent.

The invention further provides a method of evaluating the effect of a candidate pharmaceutical drug on obesity or hyperinsulinemia comprising administering said drug to a non-human binary transgenic animal that expresses dkk-1 nucleic acid and determining the effect of the drug on an obesity-determining property or on the level of insulin in said animal. Preferably, the animal is a rodent, more preferably a mouse or rat, and most preferably a mouse.

The invention also provides a non-human transgenic animal that overexpresses dkk-1 nucleic acid. Preferably, the animal is a rodent, most preferably a mouse.

The invention also provides a method for repairing or regenerating muscle in a mammal comprising administering to the mammal an effective amount of an antagonist to Dkk-1, preferably an antibody that binds to Dkk-1. Preferably, the mammal is human and/or the antibody is a monoclonal antibody.

The invention additionally involves a kit for repairing or regeneration muscle, said kit comprising:

(a) a container comprising an antagonist to Dkk-1, preferably an antibody that binds Dkk-1; and

(b) instructions for using the antagonist to repair or regenerate muscle in a mammal.

Therefore, the present invention provides for treatment and diagnosis of insulin resistance, hyperinsulinemia, hypoinsulinemia, and obesity and muscle repair or regeneration. The treatment regimen for obesity with Dkk-1 is expected to be useful in returning the body weight of obese subjects toward a normal, ideal body weight, as a therapy for obesity expected to result in maintenance of the lowered body weight for an extended period of time, and/or as a preventative of obesity.

### BRIEF DESCRIPTION OF THE DRAWINGS

FIG. 1 shows the relative expression levels of Dkk-1 in various adult human tissues.

FIG. 2 shows a gel of human Dkk-1 expressed in baculovirus and its clipping.

FIG. 3A shows the effects of human Dkk-1 (dark bars) on basal glucose uptake in L6 muscle cells for 2, 6, and 26 hours. FIGS. 3B and 3C show, respectively, the effects of human Dkk-1 on basal (light bars) and 30 nM-insulin-stimulated (dark bars) glucose uptake in L6 muscle cells.

FIG. 4A shows the effects of human Dkk-1 (dark bars) on basal and insulin-dependent glucose uptake at different stages of differentiation. FIG. 4B shows the effects of human Dkk-1 on basal and insulin-dependent glucose uptake (expressed as percent control) as a function of human Dkk-1 concentration (nM) upon 48-hour treatment.

FIGS. 5A-5B show respectively the effect of human Dkk-1 on the incorporation of glucose into glycogen in L6 muscle cells with (dark bars) and without (light bars) insulin for 48 hours (FIG. 5A) and 96 hours (FIG. 5B).

FIGS. 6A-6E show the effects of 40 nM human Dkk-1 on the expression levels of MyoD (FIG. 6A), MLC 2 (FIG. 6B), myosin heavy chain (FIG. 6C), myogenin (FIG. 6D), and Pax3 (FIG. 6E) in L6 muscle cells. Diamonds are control and squares are Dkk-l. One asterisk is p<0.01 and two asterisks is p<0.005, n=3.

FIG. 7 shows the effect of human Dkk-1 on expression of various genes in the insulin-signaling pathway in L6 muscle cells on day 5 (light bars) and day 7 (dark bars).

FIGS. 8A-8D show the effect of 40 nM human Dkk-1 (dark bars) on the kinase activities of PDK-1 (FIG. 8A), GSK3β (FIG. 8B), S6 kinase (FIG. 8C), and Akt (FIG. 8D) in L6 muscle cells after 48 hours of treatment with no insulin stimulation or stimulated with 1 nM insulin.

FIGS. 9A and 9B show the effect of human Dkk-1 on levels of basal (light bars) and 30 nM-insulin-stimulated (dark bars) glucose uptake of 3T3 L1 cells (adipocytes) after 48-hour and 96-hour treatment, respectively, and FIGS. 9C and 9D show the effect of human Dkk-1 on incorporation of glucose into lipids following insulin stimulation, after 48-hour treatment and 96-hour treatment, respectively.

FIGS. 10A-10D show the relative levels of PPARγ, C/EBPα, AP2, and fatty acid synthase (FAS) transcripts, respectively, in human Dkk-1-treated 3T3 L1 cells during adipocyte differentiation, with dark diamonds being control and light squares being Dkk-1.

FIG. 11A shows the level of blood glucose as a function of time post glucose bolus for female FVB mice intravenously injected with saline (diamonds) and 0.2 mg/kg human Dkk-1 (triangles). FIG. 11B shows the insulin levels in the female FVB mice intravenously injected with saline (control), 0.05 mg/kg/day human Dkk-1, and 0.2 mg/kg/day human Dkk-1.

FIG. 12A shows the effects of human Dkk-1 on expression of various markers of muscle differentiation in mice injected therewith, with control (light bars) and 0.2 mg/kg/day of human Dkk-1 (dark bars). FIG. 12B shows the amount of phosphorylated peptide in mice intravenously injected with no insulin, 33 nM insulin, and 100 nM insulin, with control being light bars (n—4) and human Dkk-1 being dark bars (n=5).

FIG. 13A shows the body weights of newborn/young male and female control mice (light bars) and Dkk-1 transgenic mice (dark bars). FIG. 13B shows the growth curves of control (C) and transgenic (TG) female and male mice on a regular diet, with female (C) diamonds, female (TG) squares, male (C) triangles, and male (TG) circles.

FIGS. 14A and 14B show the weight of fat pads for male and female control (light bars) and transgenic (dark bars) mice, respectively. FIGS. 14C and 14D show serum levels of basal and fasting leptin in transgenic and control male and female mice.

FIG. 15A shows growth curves for female control mice (diamond), male control mice (triangles), female transgenic mice (squares), and male transgenic mice (circles). FIGS. 15B and C show the weights of fat pads of male and female control (light bars) and transgenic (dark bars) mice, respectively. FIG. 15D shows non-fasting leptin levels of female and male control (light bars) and Dkk-1-treated (dark bars) mice.

FIGS. 16A and 16B show the blood glucose levels in male and female mice, respectively, as a function of time post glucose bolus, with diamonds being MDKK-1 mice and triangles being control mice in FIG. 16A and squares being control mice in FIG. 16B. FIGS. 16C and 16D show the insulin tolerance in control and Dkk-1 transgenic female and male mice, respectively, with diamonds being female control, squares being female transgenic, triangles being male control, and circles being male transgenic mice. FIG. 16E shows the glucose-induced serum insulin levels in transgenic and control mice, with light bars being female and dark bars being male mice.

FIG. 17 shows the effect of an anti-human Dkk-1 monoclonal antibody on the Dkk-1-mediated decrease in glucose uptake in L6 cells in the absence and presence of insulin, where the control L6 cells are light bars, the L6 cells with 40 nM Dkk-1 are black bars, and the L6 cells with 40 nM Dkk-1 and 0.5 μg/mL anti-Dkk-1 antibody are dark gray bars on the far right.

### DETAILED DESCRIPTION OF THE PREFERRED EMBODIMENTS

Definitions

“Insulin resistance” or an “insulin-resistant disorder” or an “insulin-resistant activity” is a disease, condition, or disorder resulting from a failure of the normal metabolic response of peripheral tissues (insensitivity) to the action of exogenous insulin, i.e., it is a condition where the presence of insulin produces a subnormal biological response. In clinical terms, insulin resistance is present when normal or elevated blood glucose levels persist in the face of normal or elevated levels of insulin. It represents, in essence, a glycogen synthesis inhibition, by which either basal or insulin-stimulated glycogen synthesis, or both, are reduced below normal levels. Insulin resistance as used herein includes abnormal glucose tolerance, Type A diabetes, and Type 2 diabetes, but not obesity that is unassociated with insulin resistance.

“Hypoinsulinemia” is a condition wherein lower than normal amounts of insulin circulate throughout the body and wherein obesity is generally not involved. This condition includes Type I diabetes.

“Diabetes mellitus” is encompassed within insulin resistance and hypoinsulinemia and refers to a state of chronic hyperglycemia, i.e., excess sugar in the blood, consequent upon a relative or absolute lack of insulin action. There are three basic types of diabetes mellitus, Type I or insulin-dependent diabetes mellitus (IDDM), Type 2 or non-insulin-dependent diabetes mellitus (NIDDM), and Type A insulin resistance, although Type A is relatively rare. Patients with either Type I or Type 2 diabetes can become insensitive to the effects of exogenous insulin through a variety of mechanisms. Type A insulin resistance results from either mutations in the insulin receptor gene or defects in post-receptor sites of action critical for glucose metabolism. Diabetic subjects can be easily recognized by the physician, and are characterized by fasting hyperglycemia, impaired glucose tolerance, glycosylated hemoglobin, and, in some instances, ketoacidosis associated with trauma or illness.

“Non-insulin dependent diabetes mellitus” or “NIDDM” refers to Type 2 diabetes. NIDDM patients have an abnormally high blood glucose concentration when fasting and delayed cellular uptake of glucose following meals or after a diagnostic test known as the glucose tolerance test. NIDDM is diagnosed based on recognized criteria (American Diabetes Association, Physician's Guide to Insulin-Dependent (Type I) Diabetes, 1988; American Diabetes Association, Physician's Guide to Non-Insulin-Dependent (Type II) Diabetes, 1988).

“Hyperinsulinemia” as used herein refers to a condition wherein higher than normal amounts of insulin circulate throughout the body, and which does not involve and is not caused by insulin resistance.

As used herein, “obesity” refers to a condition whereby a mammal has a Body Mass Index (BMI), which is calculated as weight (kg) per height² (meters), of at least 25.9. Conventionally, those persons with normal weight have a BMI of 19.9 to less than 25.9. The obesity herein may be due to any cause, whether genetic or environmental. Examples of disorders that may result in obesity or be the cause of obesity include overeating and bulimia, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. An “obesity-determining property” includes fat cells and tissue, such as fat pads, total body weight, triglyceride levels in muscle, liver and fat and fasting and non-fasting levels of leptin, free fatty acids and triglycerides in the blood.

“Repairing” or “regenerating” muscle refers to muscle tissue being at least partially healed or restored to its former healthier condition and/or function after any trauma, degeneration, and/or wasting thereof from whatever cause.

The term “mammal” for the purposes of treatment refers to any animal classified as a mammal, including but not limited to, humans, sport, zoo, pet and domestic or farm animals such as dogs, cats, cattle, sheep, pigs, horses, and non-human primates, such as monkeys. Preferably the mammal is a human, also called herein a patient.

As used herein, “treating” describes the management and care of a mammal for the purpose of combating insulin resistance, hyperinsulinemia, hypoinsulinemia, or obesity and includes administration to prevent the onset of the symptoms or complications, alleviate the symptoms or complications of, or eliminate the insulin resistance, hyperinsulinemia, hypoinsulinemia, or obesity, or to repair and/or regenerate muscle.

For purposes of this invention, beneficial or desired clinical “treatment” results for reducing insulin resistance include, but are not limited to, alleviation of symptoms associated with insulin resistance, diminishment of the extent of the symptoms of insulin resistance, stabilization (i.e., not worsening) of the symptoms of insulin resistance (e.g., reduction of insulin requirement), increase in insulin sensitivity and/or insulin secretion to prevent islet cell failure, and delay or slowing of insulin-resistance progression, e.g., diabetes progression.

Symptoms and complications of diabetes to be “treated” include hyperglycemia, unsatisfactory glycemic control, ketoacidosis, insulin resistance, elevated growth hormone levels, elevated levels of glycosylated hemoglobin and advanced glycosylation end-products (AGE), dawn phenomenon, unsatisfactory lipid profile, vascular disease (e.g., atherosclerosis), microvascular disease, retinal disorders (e.g., proliferative diabetic retinopathy), renal disorders, neuropathy, complications of pregnancy (e.g., premature termination and birth defects) and the like. Included in the definition of treatment are such end points as, for example, increase in insulin sensitivity, reduction in insulin dosing while maintaining glycemic control, decrease in HbA 1c, improved glycemic control, reduced vascular, renal, neural, retinal, and other diabetic complications, prevention or reduction of the “dawn phenomenon”, improved lipid profile, reduced complications of pregnancy, and reduced ketoacidosis. As will be understood by one of skill in the art, the particular symptoms that yield to treatment in accordance with the invention will depend on the type of insulin resistance being treated.

In the context of muscle repair and regeneration, “treatment” relates to the alleviation of muscle atrophy or trauma or degeneration and improvement in repair and/or function of the muscle tissue.

As to hyperinsulinemia or hypoinsulinemia, “treatment” refers to lowering or raising, respectively, the levels of circulating insulin in the body to acceptable or normal levels, which are defined as the general levels in a body before the mammal had the condition.

As to obesity, “treatment” generally refers to reducing the BMI of the mammal to less than about 25.9, and maintaining that weight for at least 6 months. The treatment suitably results in a reduction in food or caloric intake by the mammal. In addition, treatment in this context refers to preventing obesity from occurring if the treatment is administered prior to the onset of the obese condition. Treatment includes the inhibition and/or complete suppression of lipogenesis in obese mammals, i.e., the excessive accumulation of lipids in fat cells, which is one of the major features of human and animal obesity, as well as loss of total body weight.

Those “in need of treatment” include mammals already having the disorder, as well as those prone to having the disorder, including those in which the disorder is to be prevented.

An “insulin-resistance-treating agent” is an agent other than an antagonist to Dkk-1 that is used to treat insulin resistance, such as, for example, hypoglycemic agents. Examples of such treating agents include insulin (one or more different insulins); insulin mimetics such as a small-molecule insulin, e.g., L-783,281; insulin analogs (e.g., HUMALOG® insulin (Eli Lilly Co.), Lys^{B28}insulin, Pro^{B29}insulin, or Asp^{B21}insulin or those described in, for example, U.S. Pat. Nos. 5,149,777 and 5,514,646), or physiologically active fragments thereof; insulin-related peptides (C-peptide, GLP-1, insulin-like growth factor-I (IGE-1), or IGF-1/IGFBP-3 complex) or analogs or fragments thereof; ergoset; pramlintide; leptin; BAY-27-9955; T-1095; antagonists to insulin receptor tyrosine kinase inhibitor; antagonists to TNF-alpha function; a growth-hormone releasing agent; amylin or antibodies to amylin; an insulin sensitizer, such as compounds of the glitazone family, including those described in U.S. Pat. No. 5,753,681, such as troglitazone, pioglitazone, englitazone, and related compounds; Linalol alone or with Vitamin E (U.S. Pat. No. 6,187,333); insulin-secretion enhancers such as nateglinide (AY-4166), calcium (2S)-2-benzyl-3-(cis-hexahydro-2-isoindolinylcarbonyl)propionate dihydrate (mitiglinide, KAD-1229), and repaglinide; sulfonylurea drugs, for example, acetohexamide, chlorpropamide, tolazamide, tolbutamide, glyclopyramide and its ammonium salt, glibenclamide, glibomuride, gliclazide, 1-butyl-3-metanilylurea, carbutamide, glipizide, gliquidone, glisoxepid, glybuthiazole, glibuzole, glyhexamide, glymidine, glypinamide, phenbutamide, tolcyclamide, glimepiride, etc.; biguanides (such as phenformin, metformin, buformin, etc.); α-glucosidase inhibitors (such as acarbose, voglibose, miglitol, emiglitate, etc.), and such non-typical treatments as pancreatic transplant or autoimmune reagents.

A “weight-loss agent” refers to a molecule useful in treatment or prevention of obesity. Such molecules include, e.g., hormones (catecholamines, glucagon, ACTH, and growth hormone combined with IGF-1); the Ob protein; clofibrate; halogenate; cinchocaine; chlorpromazine; appetite-suppressing drugs acting on noradrenergic neurotransmitters such as mazindol and derivatives of phenethylamine, e.g., phenylpropanolamine, diethylpropion, phentermine, phendimetrazine, benzphetamine, amphetamine, methamphetamine, and phenmetrazine; drugs acting on serotonin neurotransmitters such as fenfluramine, tryptophan, 5-hydroxytryptophan, fluoxetine, and sertraline; centrally active drugs such as naloxone, neuropeptide-Y, galanin, corticotropin-releasing hormone, and cholecystokinin; a cholinergic agonist such as pyridostigmine; a sphingolipid such as a lysosphingolipid or derivative thereof; thermogenic drugs such as thyroid hormone; ephedrine; beta-adrenergic agonists; drugs affecting the gastrointestinal tract such as enzyme inhibitors, e.g. tetrahydrolipostatin, indigestible food such as sucrose polyester, and inhibitors of gastric emptying such as threo-chlorocitric acid or its derivatives; β-adrenergic agonists such as isoproterenol and yohimbine; aminophylline to increase the β-adrenergic-like effects of yohimbine, an α₂-adrenergic blocking drug such as clonidine alone or in combination with a growth-hormone releasing peptide; drugs that interfere with intestinal absorption such as biguanides such as metformin and phenformin; bulk fillers such as methylcellulose; metabolic blocking drugs such as hydroxycitrate; progesterone; cholecystokinin agonists; small molecules that mimic ketoacids; agonists to corticotropin-releasing hormone; an ergot-related prolactin-inhibiting compound for reducing body fat stores (U.S. Pat. No. 4,783,469 issued Nov. 8, 1988); beta-3-agonists; bromocriptine; antagonists to opioid peptides; antagonists to neuropeptide Y; glucocorticoid receptor antagonists; growth hormone agonists; combinations thereof; etc.

As used herein, “insulin” refers to any and all substances having an insulin action, and exemplified by, for example, animal insulin extracted from bovine or porcine pancreas, semi-synthesized human insulin that is enyzmatically synthesized from insulin extracted from porcine pancreas, and human insulin synthesized by genetic engineering techniques typically using E. coli or yeasts, etc. Further, insulin can include insulin-zinc complex containing about 0.45 to 0.9 (w/w)% of zinc, protamine-insulin-zinc produced from zinc chloride, protamine sulfate and insulin, etc. Insulin may be in the form of its fragments or derivatives, e.g., INS-1. Insulin may also include insulin-like substances such as L83281 and insulin agonists. While insulin is available in a variety of types such as super immediate-acting, immediate-acting, bimodal-acting, intermediate-acting, long-acting, etc., these types can be appropriately selected according to the patient's condition.

As used herein, “Dkk-1” or “Dickkopf-1” refers to Wnt inhibitor with properties and characteristics described in WO 99/46281 published Sep. 16, 1999 and Glinka et al., Nature, 391:357-62 (1998). In WO 99/46281, human Dkk-1 is designated PRO1008, and the DNA encoding it is designated DNA57530. This invention contemplates any mammalian species of native-sequence Dkk-1, including rodent, ovine, bovine, porcine, equestrian, canine, feline, non-human primate, and human Dkk-1, especially human Dkk-1. It also contemplates antagonists to any mammalian species of native-sequence Dkk-1, but preferably contemplates antagonists to rodent, ovine, bovine, porcine, canine, feline, equestrian, non-human primate, or human Dkk-1, most preferably antagonists to human Dkk-1.

A “therapeutic composition,” as used herein, is defined as comprising Dkk-1 or a Dkk-1 antagonist and a pharmaceutically acceptable carrier, such as water, minerals, proteins, and other excipients known to one skilled in the art.

The expressions, “antagonist,” “antagonist to Dkk-1,” and the like within the scope of the present invention are meant to include any molecule that interacts with Dkk-1 and interferes with its function or blocks or neutralizes a relevant activity of Dkk-1, by whatever means, depending on the indication being treated. It may prevent the interaction between Dkk-1 and one or more of its receptors. Such agents accomplish this effect in various ways. For instance, the class of antagonists that “neutralize” a Dkk-1 activity will bind to Dkk-1 with sufficient affinity and specificity to interefere with Dkk-1 as defined below. An antibody “that binds” Dkk-1 is one capable of binding that antigen with sufficient affinity such that the antibody is useful as a therapeutic agent in targeting a cell expressing the Dkk-1.

Included within this group of antagonists are, for example, antibodies directed against Dkk-1 or portions thereof reactive with Dkk-1, the Dkk-1 receptor or portions thereof reactive with Dkk-1, or any other ligand that binds to Dkk-1. The term also includes any agent that will interfere in the overproduction of dkk-1 mRNA or Dkk-1 protein or antagonize at least one Dkk-1 receptor. Such antagonists may be in the form of chimeric hybrids, useful for combining the function of the agent with a carrier protein to increase the serum half-life of the therapeutic agent or to confer cross-species tolerance. Hence, examples of such antagonists include bioorganic molecules (e.g., peptidomimetics), antibodies, proteins, peptides, glycoproteins, glycopeptides, glycolipids, polysaccharides, oligosaccharides, nucleic acids, pharmacological agents and their metabolites, transcriptional and translation control sequences, and the like. In a preferred embodiment the antagonist is an antibody having the desirable properties of binding to Dkk-1 and preventing its interaction with a receptor.

The terms “neutralize”, and “neutralize the activity of” are used herein to mean, for example, block, prevent, reduce, counteract the activity of, or make the Dkk-1 ineffective by any mechanism. Therefore, the antagonist may prevent a binding event necessary for activation of Dkk-1. By “neutralizing antibody” is meant an antibody molecule as herein defined that is able to block or significantly reduce an effector function of the Dkk-1. For example, a neutralizing antibody may inhibit or reduce the ability of Dkk-1 to interact with a Dkk-1 receptor. Alternatively, the neutralizing antibody may inhibit or reduce the ability of Dkk-1 to block the Dkk-1 receptor signalling pathway. The neutralizing antibody may also immunospecifically bind to the Dkk-1 in an immunoassay for Dkk-1 activity such as the ones described herein. It is a characteristic of the “neutralizing antibody” of the invention that it retain its functional activity in both in vitro and in vivo situations.

The term “antibody” herein is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen.

In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256: 495 (1975), or may be made by recombinant DNA methods (e g, U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate (e.g. Old World Monkey, Ape, etc.) and human constant-region sequences.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one that comprises an antigen-binding variable region as well as a light-chain constant domain (C_{L}) and heavy-chain constant domains, C_{H}1, C_{H}2 and C_{H}3. The constant domains may be native-sequence constant domains (e.g., human native-sequence constant domains) or an amino acid sequence variant thereof. Preferably, the intact antibody has one or more effector functions.

Antibody “effector functions” refer to those biological activities attributable to the Fc region (a native-sequence Fc region or amino-acid-sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down-regulation of cell-surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light-chain and the heavy-chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions.

The term “hypervariable region” when used herein refers to the amino acid residues of an antibody that are responsible for antigen-binding. The hypervariable region generally comprises amino acid residues from a “complementarity determining region” or “CDR” (e.g, residues 24-34 (L1), 50-56 (L2) and 89-97 (L3) in the light-chain variable domain and 31-35 (H1), 50-65 (H2) and 95-102 (H3) in the heavy-chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk J. Mol. Biol., 196:901-917 (1987)). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody.

However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy-chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{D} and V_{L} domains that enables the scFv to form the desired structure for antigen binding. For a review of scFv, see Plückthun in The Pharmacology of Monoclonal Antibodies, vol. 113, Rosenburg and Moore eds. (Springer-Verlag: New York, 1994), pp. 269-315.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature, 321: 522-525 (1986); Riechmann et al., Nature, 332: 323-329 (1988); and Presta, Curr. Op. Struct. Biol., 2: 593-596 (1992).

The term “sample” as used herein, refers to a biological sample containing or suspected of containing Dkk-1. This sample may come from any source, preferably a mammal and more preferably a human. Such samples include aqueous fluids such as serum, plasma, lymph fluid, synovial fluid, follicular fluid, seminal fluid, milk, whole blood, urine, cerebrospinal fluid, saliva, sputum, tears, perspiration, mucous, tissue culture medium, tissue extracts, and cellular extracts.

As used herein, the term “transgene” refers to a nucleic acid sequence that is partly or entirely heterologous, i.e., foreign, to the transgenic animal into which it is introduced, or is homologous to an endogenous gene of the transgenic animal into which it is introduced, but which is designed to be inserted, or is inserted, into the animal's genome in such a way as to alter the genome of the cell into which it is inserted (e.g., it is inserted at a location that differs from that of the natural gene). A transgene can be operably linked to one or more transcriptional regulatory sequences and any other nucleic acid, such as introns, that may be necessary for optimal expression of a selected nucleic acid. The transgene herein encodes Dkk-1.

The term “non-human transgenic animal that overexpresses dkk-1 nucleic acid” herein refers to a non-human animal, such as a rodent, that has included within a plurality of its cells the Dkk-1-encoding transgene, which alters the phenotype of the host cell with respect to glucose clearance in the blood, circulating insulin in the blood, muscle regeneration, or other properties related to insulin resistance, hypoinsulinemia, and/or muscle repair.

The term “non-human binary transgenic animal that expresses dkk-1 nucleic acid” herein refers to a non-human animal, such as a rodent, in which gene expression is controlled by the interaction of Dkk-1 on a target transgene. These interactions are controlled by crossing animal lines (such as rodent, e.g., mouse lines) or by adding or removing an exogenous inducer. Such controlled gene expression alters the phenotype of the host cell with respect to weight and fat indicators and circulating insulin in the blood, or other properties related to obesity and hyperinsulinemia.

Modes for Carrying Out the Invention

Novel methods are disclosed for diagnosing and treating insulin resistance and hypoinsulinemia based on antagonists that bind to, and preferably neutralize, the activity of Dkk-1.

Further, Dkk-1 itself is a useful treatment for obesity and hyperinsulinemia.

Additionally, antagonists to Dkk-1 are further indicated in methods herein for muscle repair and regeneration.

Therefore, the present invention provides for methods useful in a number of in vitro and in vivo diagnostic and therapeutic situations.

Dkk-1 can be obtained from any source, and may be prepared by any technique, including the methods set forth in the literature cited above, such as recombinant production or amino acid synthesis, provided it has a sequence that will be effective in treating obesity or hyperinsulinemia.

If an antagonist is indicated, it may be an antibody, preferably a monoclonal antibody, as well as a molecule capable of suppressing production of Dkk-1 or of dkk-1 mRNA. A candidate antagonist can be assayed for effectiveness, e.g., via the assay techniques as described herein, including testing the effect of the candidate antagonist on reducing circulating levels of Dkk-1 can be measured in an ELISA assay. A description follows as to exemplary techniques for the production of the antibodies used in accordance with the present invention.

Polyclonal antibodies are preferably raised in animals by multiple subcutaneous (sc) or intraperitoneal (ip) injections of the relevant antigen and an adjuvant. It may be useful to conjugate the relevant antigen to a polyhistidine tag or a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals may be immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals may be boosted with ⅕ to 1/10 the original amount of peptide or conjugate in Freund's complete adjuvant by subcutaneous injection at multiple sites. Seven to 14 days later the animals may be bled and the serum assayed for antibody titer. Animals may be boosted until the titer plateaus. In one embodiment, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler and Milstein, Nature, 256: 495-497 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107: 220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al, Curr. Opinion in Immunol., 5: 256-262 (1993) and Plückthun, Immunol. Revs., 130: 151-188 (1992).

In a further embodiment, monoclonal antibodies or antibody fragments can be isolated from antibody phage libraries generated using the techniques described in McCafferty et al., Nature, 348: 552-554 (1990). Clackson et al., Nature, 352: 624-628 (1991) and Marks et al, J. Mol. Biol., 222: 581-597 (1991) describe the isolation of murine and human antibodies, respectively, using phage libraries. Subsequent publications describe the production of high affinity (nM range) human antibodies by chain shuffling (Marks et al., Bio/Technology, 10: 779-783 (1992)), as well as combinatorial infection and in vivo recombination as a strategy for constructing very large phage libraries (Waterhouse et al., Nuc. Acids. Res., 21: 2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy-chain and light-chain constant domains in place of the homologous murine sequences (U.S. Pat. No. 4,816,567; Morrison et al., Proc. Natl. Acad. Sci. USA, 81: 6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151: 2296 (1993); Chothia et al., J. Mol. Biol., 196: 901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89: 4285 (1992); Presta et al., J. Immunol., 151: 2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of the humanized antibody or affinity-matured antibody are contemplated. For example, the humanized antibody or affinity-matured antibody may be an antibody fragment, such as a Fab, that is optionally conjugated with one or more targeting agent(s) in order to generate an immunoconjugate. Alternatively, the humanized antibody or affinity-matured antibody may be an intact antibody, such as an intact IgG1 antibody.

As an alternative to humanization, human antibodies can be generated. For example, transgenic animals (e.g., mice) may be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge (Jakobovits et al., Proc. Natl. Acad. Sci. USA, 90: 2551 (1993); Jakobovits et al., Nature, 362: 255-258 (1993); Bruggermann et al., Year in Immuno., 7: 33 (1993); and U.S. Pat. Nos. 5,591,669, 5,589,369 and 5,545,807).

Alternatively, phage display technology (McCafferty et al, Nature, 348: 552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson and Chiswell, Current Opinion in Structural Biology, 3: 564-571 (1993). Several sources of V-gene segments can be used for phage display. Clackson et al., Nature, 352: 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol., 222: 581-597 (1991), Griffith et al, EMBO J., 12: 725-734 (1993) or U.S. Pat. Nos. 5,565,332 or 5,573,905.

Human antibodies may also be generated by in vitro activated B cells (U.S. Pat. Nos. 5,567,610 and 5,229,275).

Various techniques have been developed for the production of antibody fragments. Traditionally, these fragments were derived via proteolytic digestion of intact antibodies (Morimoto et al., Journal of Biochemical and Biophysical Methods, 24: 107-117 (1992); Brennan et al., Science, 229: 81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology, 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv) (WO 93/16185; U.S. Pat. Nos. 5,571,894 and 5,587,458). The antibody fragment may also be a “linear antibody”, e.g., as described in U.S. Pat. No. 5,641,870. Such linear antibody fragments may be monospecific or bispecific.

Bispecific antibodies are antibodies that have binding specificities for at least two different epitopes. Exemplary bispecific antibodies may bind to two different epitopes of the Dkk-1 protein. Bispecific antibodies can be prepared as full-length antibodies or antibody fragments (e.g. F(ab′)₂ bispecific antibodies).

Methods for making bispecific antibodies are known in the art. Traditional production of full-length bispecific antibodies is based on the co-expression of two immunoglobulin heavy chain-light chain pairs, where the two chains have different specificities (Milstein et al., Nature, 305: 537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10: 3655-3659 (1991).

According to a different approach, antibody variable domains with the desired binding specificities (antibody-antigen combining sites) are fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121: 210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91100360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′-SH fragments can be directly recovered from E. coli and chemically coupled to form bispecific antibodies (Shalaby et al., J. Exp. Med., 175: 217-225 (1992)).

Various techniques for making and isolating bispecific antibody fragments directly from recombinant cell culture have also been described. For example, bispecific antibodies have been produced using leucine zippers (Kostelny et al., J. Immunol., 148: 1547-1553 (1992)). The leucine zipper peptides from the Fos and Jun proteins are linked to the Fab′ portions of two different antibodies by gene fusion. The antibody homodimers are reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90: 6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported (Gruber et al., J. Immunol., 152: 5368 (1994)).

Antibodies with more than two valencies are contemplated. For example, trispecific antibodies can be prepared (Tutt et al., J. Immunol., 147: 60 (1991)).

Amino acid sequence modification(s) of the anti-Dkk-1 antibodies described herein are contemplated. For example, it may be desirable to improve the binding affinity and/or other biological properties of the antibody. Amino acid sequence variants of the anti-Dkk-1 antibody are prepared by introducing appropriate nucleotide changes into the anti-Dkk-1 antibody nucleic acid, or by peptide synthesis. Such modifications include, for example, deletions from, and/or insertions into and/or substitutions of, residues within the amino acid sequences of the anti-Dkk-1 antibody. Any combination of deletion, insertion, and substitution is made to arrive at the final construct, provided that the final construct possesses the desired characteristics. The amino acid changes also may alter post-translational processes of the anti-Dkk-1 antibody, such as changing the number or position of glycosylation sites.

A useful method for identification of certain residues or regions of the anti-Dkk-1 antibody that are preferred locations for mutagenesis is “alanine scanning mutagenesis” (Cunningham and Wells, Science, 244:1081-1085 (1989)). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with Dkk-1 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed anti-Dkk-1 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-Dkk-1 antibody with an N-terminal methionyl residue or the antibody fused to a hypoglycemic polypeptide. Other insertional variants of the anti-Dkk-1 antibody molecule include the fusion to the N- or C-terminus of the anti-Dkk-1 antibody to an enzyme or a polypeptide that increases the serum half-life of the antibody.

Another type of variant is an amino acid substitution variant. These variants have at least one amino acid residue in the anti-Dkk-1 antibody molecule replaced by a different residue. The sites of greatest interest for substitutional mutagenesis include the hypervariable regions, but FR alterations are also contemplated. Conservative substitutions are shown in Table I under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the anti-Dkk-1 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and Dkk-1. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-Dkk-1 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, or cassette mutagenesis of an earlier prepared variant or a non-variant version of the anti-Dkk-1 antibody.

It may be desirable to modify the antibody of the invention with respect to effector function, e.g., so as to enhance Fc receptor binding. This may be achieved by introducing one or more amino acid substitutions into an Fc region of the antibody. Alternatively or additionally, cysteine residue(s) may be introduced in the Fc region, thereby allowing interchain disulfide bond formation in this region.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol.

Therapeutic Uses for Muscle, Insulin-Resistance, and Hypoinsulinemia Indications

For the muscle, insulin-resistant, and hypoinsulinemic indications, the Dkk-1 antagonist is administered by any suitable route, including a parenteral route of administration such as, but not limited to, intravenous (IV), intramuscular (IM), subcutaneous (SC), and intraperitoneal (IP), as well as transdermal, buccal, sublingual, intrarectal, intranasal, and inhalant routes. IV, IM, SC, and IP administration may be by bolus or infusion, and in the case of SC, may also be by slow-release implantable device, including, but not limited to pumps, slow-release formulations, and mechanical devices. Preferably, administration is systemic.

One specifically preferred method for administration of Dkk-1 antagonist is by subcutaneous infusion, particularly using a metered infusion device, such as a pump. Such pump can be reusable or disposable, and implantable or externally mountable. Medication infusion pumps that are usefully employed for this purpose include, for example, the pumps disclosed in U.S. Pat. Nos. 5,637,095; 5,569,186; and 5, 527,307. The compositions can be administered continaully from such devices, or intermittently.

Therapeutic formulations of Dkk-1 antagonists suitable for storage include mixtures of the antagonist having the desired degree of purity with pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENT™, PLURONICS™ or polyethylene glycol (PEG). Preferred lyophilized anti-Dkk-1 antibody formulations are described in WO 97/04801. These compositions comprise antagonist to Dkk-1 containing from about 0.1 to 90% by weight of the active antagonist, preferably in a soluble form, and more generally from about 10 to 30%.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

The anti-Dkk-1 antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide interchange reaction.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Any of the specific antagonists can be joined to a carrier protein to increase the serum half-life of the therapeutic antagonist. For example, a soluble immunoglobulin chimera, such as described herein, can be obtained for each specific Dkk-1 antagonist or antagonistic portion thereof, as described in U.S. Pat. No. 5,116,964. The immunoglobulin chimera are easily purified through IgG-binding protein A-Sepharose chromatography. The chimera have the ability to form an immunoglobulin-like dimer with the concomitant higher avidity and serum half-life.

The formulations to be used for in vivo administration must be sterile. This is readily accomplished by filtration through sterile filtration membranes.

The formulation herein may also contain more than one active compound as necessary for the particular indication being treated, preferably those with complementary activities that do not adversely affect each other. Also, such active compound can be administered separately to the mammal being treated.

For example, it may be desirable to further provide an insulin-resistance-treating agent for those indications. In addition, Type 2 diabetics that fail to respond to diet and weight loss may respond to therapy with sulfonylureas along with the Dkk-1 antagonist. The class of sulfonylurea drugs includes acetohexamide, chlorpropamide, tolazamide, tolbutamide, glibenclaminde, glibomuride, gliclazide, glipizide, gliquidone and glymidine. Other agents for this purpose include an autoimmune reagent, an insulin sensitizer, such as compounds of the glitazone family, including those described in U.S. Pat. No. 5,753,681, such as troglitazone, pioglitazone, englitazone, and related compounds, antagonists to insulin receptor tyrosine kinase inhibitor (U.S. Pat. Nos. 5,939,269 and 5,939,269), IGF-1/IGFBP-3 complex (U.S. Pat. No. 6,040,292), antagonists to TNF-alpha function (U.S. Pat. No. 6,015,558), growth hormone releasing agent (U.S. Pat. No. 5,939,387), and antibodies to amylin (U.S. Pat. No. 5,942,227). Other compounds that can be used include insulin (one or more different insulins), insulin mimetics such as a small-molecule insulin, insulin analogs as noted above or physiologically active fragments thereof, insulin-related peptides as noted above, or analogs or fragments thereof. Agents are further specified in the definition above.

For treating hypoinsulinemia, for example, insulin may be administered together or separately from the antagonist to Dkk-1.

Such additional molecules are suitably present or administered in combination in amounts that are effective for the purpose intended, typically less than what is used if they are administered alone without the antagonist to Dkk-1. If they are formulated together, they may be formulated in the amounts determined according to, for example, the type of indication, the subject, the age and body weight of the subject, current clinical status, administration time, dosage form, administration method, etc. For instance, a concomitant drug is used preferably in a proportion of about 0.0001 to 10,000 weight parts relative to one weight part of the antagonist to Dkk-1 herein.

Use of the antagonist to Dkk-1 in combination with insulin enables reduction of the dose of insulin as compared with the dose at the time of administration of insulin alone. Therefore, risk of blood vessel complication and hypoglycemia induction, both of which may be problems with large amounts of insulin administration, is low. For administration of insulin to an adult diabetic patient (body weight about 50 kg), for example, the dose per day is usually about 10 to 100 U (Units), preferably 10 to 80 U, but this may be less as determined by the physician. For administration of insulin secretion enhancers to the same type of patient, for example, the dose per day is preferably about 0.1 to 1000 mg, more preferably about 1 to 100 mg. For administration of biguanides to the same type of patient, for example, the dose per day is preferably about 10 to 2500 mg, more preferably about 100 to 1000 mg. For administration of a-glucosidase inhibitors to the same type of patient, for example, the dose per day is preferably about 0.1 to 400 mg, more preferably about 0.6 to 300 mg. Administration of ergoset, pramlintide, leptin, BAY-27-9955, or T-1095 to such patients can be effected at a dose of preferably about 0.1 to 2500 mg, more preferably about 0.5 to 1000 mg. All of the above doses can be administered once to several times a day.

The Dkk-1 antagonist may also be administered together with a suitable non-drug treatment for insulin resistance such as a pancreatic transplant.

The dosages of antagonist administered to an insulin-resistant or hypoinsulinemic mammal will be determined by the physician in the light of the relevant circumstances, including the condition of the mammal, the type of antagonist, the type of indication, and the chosen route of administration. The dosage is preferably at a sufficiently low level as not to cause weight gain to any significant degree, and the physician can determine that level. Glitazones approved for the treatment of human Type 2 diabetes (rosiglitazone/Avandia and pioglitazone/Actos) cause some weight gain, yet they are used despite the side effects because they have proven to be beneficial by their therapeutic index. The dosage ranges presented herein are not intended to limit the scope of the invention in any way. A “therapeutically effective” amount for purposes herein for hypoinsulinemia and insulin resistance is determined by the above factors, but is generally about 0.01 to 100 mg/kg body weight/day. The preferred dose is about 0.1-50 mg/kg/day, more preferably about 0.1 to 25 mg/kg/day. More preferred still, when the Dkk-1 antagonist is administered daily, the intravenous or intramuscular dose for a human is about 0.3 to 10 mg/kg of body weight per day, more preferably, about 0.5 to 5 mg/kg. For subcutaneous administration, the dose is preferably greater than the therapeutically-equivalent dose given intravenously or intramuscularly. Preferably, the daily subcutaneous dose for a human is about 0.3 to 20 mg/kg, more preferably about 0.5 to 5 mg/kg for both indications.

The invention contemplates a variety of dosing schedules. The invention encompasses continuous dosing schedules, in which Dkk-1 antagonist is administered on a regular (daily, weekly, or monthly, depending on the dose and dosage form) basis without substantial breaks. Preferred continuous dosing schedules include daily continuous infusion, where Dkk-1 antagonist is infused each day, and continuous bolus administration schedules, where Dkk-1 antagonist is administered at least once per day by bolus injection or inhalant or intranasal routes. The invention also encompasses discontinuous dosing schedules. The exact parameters of discontinuous administration schedules will vary according to the formulation, method of delivery, and clinical needs of the mammal being treated. For example, if the Dkk-1 antagonist is administered by infusion, administration schedules may comprise a first period of administration followed by a second period in which Dkk-1 antagonist is not administered that is greater than, equal to, or less than the first period.

Where the administration is by bolus injection, especially bolus injection of a slow-release formulation, dosing schedules may also be continuous in that Dkk-1 antagonist is administered each day, or may be discontinuous, with first and second periods as described above.

Continuous and discontinuous administration schedules by any method also include dosing schedules in which the dose is modulated throughout the first period, such that, for example, at the beginning of the first period, the dose is low and increased until the end of the first period, the dose is initially high and decreased during the first period, the dose is initially low, increased to a peak level, then reduced towards the end of the first period, and any combination thereof.

The effects of administration of Dkk-1 antagonist on insulin resistance can be measured by a variety of assays known in the art. Most commonly, alleviation of the effects of diabetes will result in improved glycemic control (as measured by serial testing of blood glucose), reduction in the requirement for insulin to maintain good glycemic control, reduction in glycosylated hemoglobin, reduction in blood levels of advanced glycosylation end-products (AGE), reduced “dawn phenomenon”, reduced ketoacidosis, and improved lipid profile. Alternately, administration of Dkk-1 antagonist can result in a stabilization of the symptoms of diabetes, as indicated by reduction of blood glucose levels, reduced insulin requirement, reduced glycosylated hemoglobin and blood AGE, reduced vascular, renal, neural and retinal complications, reduced complications of pregnancy, and improved lipid profile.

The blood sugar lowering effect of the Dkk-1 antagonist can be evaluated by determining the concentration of glucose or Hb (hemoglobin)A_{1c} in venous blood plasma in the subject before and after administration, and then comparing the obtained concentration before administration and after administration. HbA_{1c} means glycosylated hemoglobin, and is gradually produced in response to blood glucose concentration. Therefore, HbA_{1c} is thought important as an index of blood sugar control that is not easily influenced by rapid blood sugar changes in diabetic patients.

Evidence of treating hypoinsulinemia is shown, for example, by an increase in circulating levels of insulin in the patient.

The dosing for muscle repair and regeneration is typically about 0.01 to 100 mg/kg body weight, more preferably 1 to 10 mg/kg depending on the patient's condition, the specific type of muscle repair desired, etc. The dosing schedule is in accordance with the standard schedule used by a clinician in this area. Evidence of muscle repair or regeneration is shown by various measurement tests well known in the art, including assaying for proliferation and differentiation of muscle cells and a polymerase chain reaction test (see, e.g., Best et al., J. Orthop. Res., 19: 565-572 (2001), which provides an analysis of changes in mRNA levels of myoblast- and fibroblast-derived gene products in healing rabbit skeletal muscle using quantitative reverse transcription-polymerase chain reaction).

The invention also provides kits for the treatment of insulin resistance and hypoinsulinemia, and for repair and regeneration of muscle. The kits of the invention comprise one or more containers of Dkk-1 antagonist, preferably antibody, in combination with a set of instructions, generally written instructions, relating to the use and dosage of Dkk-1 antagonist for the treatment of insulin resistance or hypoinsulinemia, or for repair or regeneration of muscle. The instructions included with the kit generally include information as to dosage, dosing schedule, and route of administration for the treatment of the insulin-resistant or hypoinsulinemic disorder or muscle condition. The containers of Dkk-1 antagonist may be unit doses, bulk packages (e.g., multi-dose packages), or sub-unit doses.

Dkk-1 antagonist may be packaged in any convenient, appropriate packaging. For example, if the Dkk-1 antagonist is a freeze-dried formulation, an ampoule with a resilient stopper is normally used, so that the drug may be easily reconstituted by injecting fluid through the resilient stopper. Ampoules with non-resilient, removable closures (e.g., sealed glass) or resilient stoppers are most conveniently used for injectable forms of Dkk-1 antagonist. Also contemplated are packages for use in combination with a specific device, such as an inhaler, a nasal administration device (e.g, an atomizer), or an infusion device such as a minipump.

Therapeutic Use for Obesity and Hyperinsulinemia Indications

For the obesity and hyperinsulinemia indications, the Dkk-1 is administered by any suitable route, including a parenteral route of administration such as, but not limited to, intravenous (IV), intramuscular (IM), subcutaneous (SC), and intraperitoneal (IP), as well as transdermal, buccal, sublingual, intrarectal, intranasal, and inhalant routes. IV, IM, SC, and IP administration may be by bolus or infusion, and in the case of SC, may also be by slow-release implantable device, including, but not limited to pumps, slow-release formulations, and mechanical devices. Preferably, administration is systemic.

One specifically preferred method for administration of Dkk-1 is by subcutaneous infusion, particularly using a metered infusion device, such as a pump. Such pump can be reusable or disposable, and implantable or externally mountable. Medication infusion pumps that are usefully employed for this purpose include, for example, the pumps disclosed in U.S. Pat. Nos. 5,637,095; 5,569,186; and 5, 527,307. The compositions can be administered continaully from such devices, or intermittently.

Therapeutic formulations of Dkk-1 suitable for storage include mixtures of the Dkk-1 having the desired degree of purity with pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Preferred lyophilized Dkk-1 formulations are described in WO 97/04801. These compositions comprise Dkk-1 containing from about 0.1 to 90% by weight of the active Dkk-1, preferably in a soluble form, and more generally from about 10 to 30%.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Liposome formulations of the Dkk-1 can also readily be made by conventional methods. In addition, sustained-release preparations may be prepared. Suitable examples of sustained-release preparations include semipermeable matrices of solid hydrophobic polymers containing the Dkk-1, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The Dkk-1 can be joined to a carrier protein or PEG or POG or other molecule of this nature to increase its serum half-life, as is well known to those skilled in the art.

The formulations to be used for in vivo administration must be sterile. This is readily accomplished by filtration through sterile filtration membranes.

For treatment of hyperinsulinemia, the administration of Dkk-1 may occur in conjunction with, for example, diazoxide (see, for example, Shaer, Nephron, 89: 337-339 (2001)).

For treatment of obesity, the administration of Dkk-1 may occur without, or may be imposed with, a dietary restriction such as a limit in daily food or calorie intake, as is desired for the individual patient. In addition, the Dkk-1 is appropriately administered in combination with other treatments for combatting or preventing obesity, known herein as weight-loss agents. Substances useful for this purpose include, e.g., hormones (catecholamines, glucagon, ACTH, and growth hormone combined with insulin-like growth factor); the Ob protein; clofibrate; halogenate; cinchocaine; chlorpromazine; appetite-suppressing drugs acting on noradrenergic neurotransmitters such as mazindol and derivatives of phenethylamine, e.g., phenylpropanolamine, diethylpropion, phentermine, phendimetrazine, benzphetamine, amphetamine, methamphetamine, and phenmetrazine; drugs acting on serotonin neurotransmitters such as fenfluramine, tryptophan, 5-hydroxytryptophan, fluoxetine, and sertraline; centrally active drugs such as naloxone, neuropeptide-Y, galanin, corticotropin-releasing hormone, and cholecystokinin; a cholinergic agonist such as pyridostigmine; a sphingolipid such as a lysosphingolipid or derivative thereof (EP 321,287 published Jun. 21, 1989); thermogenic drugs such as thyroid hormone; ephedrine; beta-adrenergic agonists; drugs affecting the gastrointestinal tract such as enzyme inhibitors, e.g., tetrahydrolipostatin, indigestible food such as sucrose polyester, and inhibitors of gastric emptying such as threo-chlorocitric acid or its derivatives; adrenergic agonists such as isoproterenol and yohimbine; aminophylline to increase the β-adrenergic-like effects of yohimbine, an β-adrenergic blocking drug such as clonidine alone or in combination with a growth hormone releasing peptide (U.S. Pat. No. 5,120,713 issued Jun. 9, 1992); drugs that interfere with intestinal absorption such as biguanides such as metformin and phenformin; bulk fillers such as methylcellulose; metabolic blocking drugs such as hydroxycitrate; progesterone; cholecystokinin agonists; small molecules that mimic ketoacids; agonists to corticotropin-releasing hormone; an ergot-related prolactin-inhibiting compound for reducing body fat stores (U.S. Pat. No. 4,783,469 issued Nov. 8, 1988); beta-3-agonists; bromocriptine; antagonists to opioid peptides; antagonists to neuropeptide Y; glucocorticoid receptor antagonists; growth hormone agonists; combinations thereof; etc. This includes all drugs described by Bray and Greenway, Clinics in Endocrinol. and Metabol., 5: 455 (1976).

These weight-loss adjunctive agents and diazoxide may be administered at the same time as, before, or after the administration of the Dkk-1 and can be administered by the same or a different administration route than the Dkk-1 is administered.

The dosages of Dkk-1 administered to an obese or hyperinsulinemic mammal will be determined by the physician in the light of the relevant circumstances, including the condition of the mammal, the type of Dkk-1, and the chosen route of administration. The dosage is preferably at a sufficiently low level as not to cause insulin-resistance, and the physician can determine that level. Glitazones, approved for the treatment of human Type 2 diabetes (rosiglitazone/Avandia and pioglitazone/Actos), cause some weight gain, yet they are used despite the side effects because their therapeutic index shows that they are overall beneficial. The dosage ranges presented herein are not intended to limit the scope of the invention in any way. A “therapeutically effective” amount of Dkk-1 for purposes herein is determined by the above factors, but is generally about 0.01 to 100 mg/kg body weight/day for both indications. The preferred dose is about 0.1-50 mg/kg/day, more preferably about 0.1 to 25 mg/kg/day. More preferred still, when the Dkk-1 is administered daily, the intravenous or intramuscular dose for a human is about 0.3 to 10 mg/kg of body weight per day, more preferably, about 0.5 to 5 mg/kg. For subcutaneous administration, the dose is preferably greater than the therapeutically-equivalent dose given intravenously or intramuscularly. Preferably, the daily subcutaneous dose for a human is about 0.3 to 20 mg/kg, more preferably about 0.5 to 5 mg/kg for both indications.

The invention contemplates a variety of dosing schedules. The invention encompasses continuous dosing schedules, in which Dkk-1 is administered on a regular (daily, weekly, or monthly, depending on the dose and dosage form) basis without substantial breaks. Preferred continuous dosing schedules include daily continuous infusion, where Dkk-1 is infused each day, and continuous bolus administration schedules, where Dkk-1 is administered at least once per day by bolus injection or inhalant or intranasal routes. The invention also encompasses discontinuous dosing schedules. The exact parameters of discontinuous administration schedules will vary according to the formulation, method of delivery and the clinical needs of the mammal being treated. For example, if the Dkk-1 is administered by infusion, administration schedules may comprise a first period of administration followed by a second period in which Dkk-1 is not administered that is greater than, equal to, or less than the first period.

Where the administration is by bolus injection, especially bolus injection of a slow-release formulation, dosing schedules may also be continuous in that Dkk-1 is administered each day, or may be discontinuous, with first and second periods as described above.

Continuous and discontinuous administration schedules by any method also include dosing schedules in which the dose is modulated throughout the first period, such that, for example, at the beginning of the first period, the dose is low and increased until the end of the first period, the dose is initially high and decreased during the first period, the dose is initially low, increased to a peak level, then reduced towards the end of the first period, and any combination thereof.

The effects of administration of Dkk-1 on obesity can be measured likewise by a variety of assays known in the art, including analysis of fat cells and tissue, such as fat pads, total body weight, triglyceride levels in muscle, liver, and fat, fasting and non-fasting levels of leptin, and the levels of free fatty acids and triglycerides in the blood. The effects of administration of Dkk-1 on hyperinsulinemia can be measured also by a variety of assays, the most prevalent being measuring the levels of circulating insulin in the body.

The invention also provides kits for the treatment of obesity or hyperinsulinemia. The kits of the invention comprise one or more containers of Dkk-1, preferably human Dkk-1, in combination with a set of instructions, generally written instructions, relating to the use and dosage of Dkk-1 for the treatment of obesity or hyperinsulinemia. The instructions included with the kit generally include information as to dosage, dosing schedule, and route of administration for the treatment of the obese or hyperinsulinemic condition. The containers of Dkk-1 may be unit doses, bulk packages (e.g., multi-dose packages), or sub-unit doses.

Dkk-1 may be packaged in any convenient, appropriate packaging. For example, if the Dkk-1 is a freeze-dried formulation, an ampoule with a resilient stopper is normally used, so that the drug may be easily reconstituted by injecting fluid through the resilient stopper. Ampoules with non-resilient, removable closures (e.g., sealed glass) or resilient stoppers are most conveniently used for injectable forms of Dkk-1. Also contemplated are packages for use in combination with a specific device, such as an inhaler, a nasal administration device (e g., an atomizer), or an infusion device such as a minipump.

Diagnostic Uses

Many different assays and assay formats can be used to detect the amount of Dkk-1 in a sample relative to a control sample. These formats, in turn are useful in the diagnostic assays of the present invention, which are used to detect the presence or onset of insulin resistance, hyper- or hypoinsulinemia, or obesity in a mammal.

Any procedure known in the art for the measurement of soluble analytes can be used in the practice of the instant invention. Such procedures include but are not limited to competitive and non-competitive assay systems using techniques such as radioimmunoassay, enzyme immunoassays (EIA), preferably ELISA, “sandwich” immunoassays, precipitin reactions, gel diffusion reactions, immunodiffusion assays, agglutination assays, complement-fixation assays, immunoradiometric assays, fluorescent immunoassays, protein A immunoassays, and immunoelectrophoresis assays. For examples of preferred immunoassay methods, see U.S. Pat. Nos. 4,845,026 and 5,006,459.

In one embodiment, one or more of the anti-Dkk-1 antibodies used in the assay is labeled; in another embodiment, a first is unlabeled, and a labeled, second antibody is used to detect the Dkk-1 bound to the first antibody or is used to detect the first antibody.

For diagnostic applications, the antibody typically will be labeled with a detectable moiety. Numerous labels are available which can be generally grouped into the following categories:

(a) Radioisotopes, such as ³⁵S, ¹⁴C, ¹²⁵I, ³H, and 131I. The antibody can be labeled with the radioisotope or radionuclide using the techniques described in Current Protocols in Immunology, Volumes 1 and 2, Coligen et al., Ed. (Wiley-Interscience: New York, 1991), for example, and radioactivity can be measured using scintillation counting.

(b) Fluorescent labels such as rare earth chelates (europium chelates) or fluorescein and its derivatives (such as fluorescein isothiocyanate), rhodamine and its derivatives, phycoerythrin, phycocyanin, allophycocyanin, o-phthaldehyde, fluorescamine, dansyl, lissamine, and Texas Red are available. The fluorescent labels can be conjugated to the antibody using the techniques disclosed in Current Protocols in Immunology, supra, for example. Fluorescence can be quantified using a fluorimeter. The detecting antibody can also be detectably labeled using fluorescence-emitting metals such as ¹⁵²Eu or others of the lanthanide series. These metals can be attached to the antibody using such metal-chelating groups as diethylenetriaminepentaacetic acid (DTPA) or ethylenediaminetetraacetic acid (EDTA).

(c) Various enzyme-substrate labels are available for an EIA, and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence, chemiluminescence, or bioluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, aequorin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, yeast alcohol dehydrogenase, alpha-glycerophosphate dehydrogenase, and glucose-6-phosphate dehydrogenase), staphylococcal nuclease, delta-V-steroid isomerase, triose phosphate isomerase, asparaginase, ribonuclease, urease, catalase, acetylcholinesterase, heterocyclic oxidases (such as uricase and xanthine oxidase), lactoperoxidase, microperoxidase, and the like. Techniques for conjugating enzymes to antibodies are described in O'Sullivan et al., Methods in Enzym., ed. Langone and Van Vunakis (Academic Press: New York) 73: 147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e g., digoxin) and one of the different types of labels mentioned above is conjugated with an anti-hapten antibody (e.g., anti-digoxin antibody). Thus, indirect conjugation of the label with the antibody can be achieved.

In another embodiment of the invention, the anti-Dkk-1 antibody need not be labeled, and the presence thereof can be detected using a labeled antibody which binds to the Dkk-1 antibody.

The antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

In the assays of the present invention, an antigen such as Dkk-1, or an antibody is preferably bound to a solid phase support or carrier. By “solid phase support or carrier” is intended any support capable of binding an antigen or antibodies. Well-known supports, or carriers, include glass, polystyrene, polypropylene, polyethylene, dextran, nylon, amyloses, natural and modified celluloses, polyacrylamides, agaroses, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

In a preferred embodiment, an antibody-antigen-antibody sandwich immunoassay is done, i.e., antigen is detected or measured by a method comprising binding of a first antibody to the antigen, and binding of a second antibody to the antigen, and detecting or measuring antigen immunospecifically bound by both the first and second antibody. In a specific embodiment, the first and second antibodies are monoclonal antibodies. In this embodiment, if the antigen does not contain repetitive epitopes recognized by the monoclonal antibody, the second monoclonal antibody must bind to a site different from that of the first antibody (as reflected e.g., by the lack of competitive inhibition between the two antibodies for binding to the antigen). In another specific embodiment, the first or second antibody is a polyclonal antibody. In yet another specific embodiment, both the first and second antibodies are polyclonal antibodies.

In a preferred embodiment, a “forward” sandwich enzyme immunoassay is used, as described schematically below. An antibody (capture antibody, Ab1) directed against the Dkk-1 is attached to a solid phase matrix, preferably a microplate. The sample is brought in contact with the Ab1-coated matrix such that any Dkk-1 in the sample to which Ab1 is specific binds to the solid-phase Ab1. Unbound sample components are removed by washing. An enzyme-conjugated second antibody (detection antibody, Ab2) directed against a second epitope of the antigen binds to the antigen captured by Ab1 and completes the sandwich. After removal of unbound Ab2 by washing, a chromogenic substrate for the enzyme is added, and a colored product is formed in proportion to the amount of enzyme present in the sandwich, which reflects the amount of antigen in the sample. The reaction is terminated by addition of stop solution. The color is measured as absorbance at an appropriate wavelength using a spectrophotometer. A standard curve is prepared from known concentrations of the antigen, from which unknown sample values can be determined.

Other types of “sandwich” assays are the so-called “simultaneous” and “reverse” assays. A simultaneous assay involves a single incubation step as the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays.

Kits comprising one or more containers or vials containing components for carrying out the assays of the present invention are also within the scope of the invention. Such kit is a packaged combination of reagents in predetermined amounts with instructions for performing the diagnostic assay. For instance, such a kit can comprise an antibody or antibodies, preferably a pair of antibodies to the Dkk-1 antigen that preferably do not compete for the same binding site on the antigen. In a specific embodiment, Dkk-1 may be pre-adsorbed to the solid phase matrix. The kit preferably contains the other necessary washing reagents well-known in the art. For EIA, the kit contains the chromogenic substrate as well as a reagent for stopping the enzymatic reaction when color development has occurred. The substrate included in the kit is one appropriate for the enzyme conjugated to one of the antibody preparations. These are well-known in the art, and some are exemplified below. The kit can optionally also comprise a Dkk-1 standard; i.e., an amount of purified Dkk-1 corresponding to a normal amount of Dkk-1 in a standard sample.

Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In one aspect, a kit comprises in more than one container: an antibody that binds Dkk-1, which can be coated on a solid-phase carrier, e.g., a microtiter plate, a standard sample containing Dkk-1, and instructions for use in detection, wherein the antibody that binds Dkk-1 is detectably labeled or the kit further comprises an antibody that binds Dkk-1 and is detectably labeled, or binds to the first antibody.

Transgenic and Knockout Animals and Uses Thereof to Screen

Nucleic acids that encode Dkk-1 from non-human animal species, such as rodent, and more preferably murine, can be used to generate non-human transgenic or binary transgenic animals, which, in turn, are useful in the development and screening of therapeutically useful reagents. The Dkk-1 knockout mice are embryonic lethal (Mukhopadhyay et al., Dev. Cell., 1: 423-434 (2001)).

A transgenic animal is one having cells that contain a transgene, which was introduced into the animal or an ancestor of the animal at a prenatal, e.g., an embryonic stage. A transgene is a DNA that is integrated into the genome of a cell from which a transgenic animal develops.

In one embodiment, the transgenic animals are produced by introducing the Dkk-1 transgene into the germline of the non-human animal. Methods for generating transgenic animals, particularly animals such as mice, have become conventional in the art and are described, for example, in U.S. Pat. Nos. 4,736,866 and 4,870,009. Animal cDNA such as murine cDNA encoding Dkk-1 or an appropriate sequence thereof can be used to clone genomic DNA encoding Dkk-1 in accordance with established techniques, and the genomic sequences are used to generate transgenic animals that contain cells that express DNA encoding Dkk-1. Typically, particular cells would be targeted for transgene incorporation with tissue-specific enhancers, which results in targeted overexpression of Dkk-1. Transgenic animals that include a copy of a transgene encoding Dkk-1 introduced into the germ line of the animal at an embryonic stage can be used to examine the effect of increased expression of DNA encoding Dkk-1.

Embryonic target cells at various developmental stages can be used to introduce transgenes. Different methods are used depending on the stage of development of the embryonic target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used. The line(s) used to practice this invention may themselves be transgenic animals, and/or may be knockouts (i.e., obtained from animals that have one or more genes partially or completely suppressed).

The transgene construct may be introduced into a single-stage embryo. The zygote is the best target for microinjection. The use of zygotes as a target for gene transfer has a major advantage in that in most cases the injected DNA will be incorporated into the host gene before the first cleavage (Brinster et al., Proc. Natl. Acad. Sci. USA, 82: 4438-4442 (1985)). As a consequence, all cells of the transgenic animal will carry the incorporated transgene. This will in general also be reflected in the efficient transmission of the transgene to offspring of the founder, since 50% of the germ cells will harbor the transgene.

Normally, fertilized embryos are incubated in suitable media until the pronuclei appear. At about this time, the nucleotide sequence comprising the transgene is introduced into the female or male pronucleus. In some species such as mice, the male pronucleus is preferred. The exogenous genetic material may be added to the male DNA complement of the zygote prior to its being processed by the ovum nucleus or the zygote female pronucleus.

Thus, the exogenous genetic material may be added to the male complement of DNA or any other complement of DNA prior to its being affected by the female pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Any technique that allows for the addition of the exogenous genetic material into nucleic genetic material can be utilized so long as it is not destructive to the cell, nuclear membrane, or other existing cellular or genetic structures. Introduction of the transgene nucleotide sequence into the embryo may be accomplished by any means known in the art, such as, for example, microinjection, electroporation, or lipofection. The exogenous genetic material is preferentially inserted into the nucleic genetic material by microinjection. Microinjection of cells and cellular structures is known and is used in the art. In the mouse, the male pronucleus reaches the size of approximately 20 micrometers in diameter, which allows reproducible injection of 1-2 pL of DNA solution. Following introduction of the transgene nucleotide sequence into the embryo, the embryo may be incubated in vitro for varying amounts of time, or reimplanted into the surrogate host, or both. In vitro incubation to maturity is within the scope of this invention. One common method is to incubate the embryos in vitro for about 1-7 days, depending on the species, and then reimplant them into the surrogate host.

The number of copies of the transgene constructs that are added to the zygote depends on the total amount of exogenous genetic material added and will be the amount that enables the genetic transformation to occur. Theoretically only one copy is required; however, generally numerous copies are utilized, for example, 1,000-20,000 copies of the transgene construct, to ensure that one copy is functional. As regards the present invention, there may be an advantage to having more than one functioning copy of the inserted exogenous DNA sequence to enhance the phenotypic expression thereof.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the Dkk-1 encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of blood constituents such as glucose.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with this invention will include exogenous genetic material, i.e., a DNA sequence that results in the production of Dkk-1. The sequence will be attached operably to a a transcriptional control element, e.g., promoter, which preferably allows the expression of the transgene production in a specific type of cell. The most preferred such control element herein is a muscle-specific promoter that enables overexpression of the dkk-1 nucleic acid (e.g., cDNA) in muscle tissue. An example of such promoter is that described in Example 1 below or that driving smoothelin A or B expression or similar such promoters, as described, for example, in WO 01/18048 published Mar. 15, 2001.

Retroviral infection can also be used to introduce the transgene into a non-human animal. The developing non-human embryo can be cultured in vitro to the blastocyst stage. During this time, the blastomeres can be targets for retroviral infection (Jaenich, Proc. Natl. Acad. Sci. USA, 73:1260-1264 (1976)). Efficient infection of the blastomeres is obtained by enzymatic treatment to remove the zona pellucida (Manipulating the Mouse Embryo, Hogan, ed. (Cold Spring Harbor Laboratory Press, Cold Spring Harbor, N.Y., 1986)). The viral vector system used to introduce the transgene is typically a replication-defective retrovirus carrying the transgene (Jahner et al., Proc. Natl. Acad. Sci. USA, 82: 6972-6931 (1985); Van der Putten et al., Proc. Natl. Acad. Sci. USA, 82: 6148-6152 (1985)). Transfection is easily and efficiently obtained by culturing the blastomeres on a monolayer of virus-producing cells (Van der Putten et al., supra; Stewart et al., EMBO J., 6: 383-388 (1987)). Alternatively, infection can be performed at a later stage. Virus or virus-producing cells can be injected into the blastocoele (Jahner et al., Nature, 298: 623-628 (1982)). Most of the founders will be mosaic for the transgene since incorporation occurs only in a subset of the cells that formed the transgenic non-human animal. Further, the founder may contain various retroviral insertions of the transgene at different positions in the genome that generally will segregate in the offspring. In addition, it is also possible to introduce transgenes into the germ line by intrauterine retroviral infection of the mid-gestation embryo (Jahner et al. (1982), supra).

A third type of target cell for transgene introduction is the embryonic stem cell (ES). ES cells are obtained from pre-implantation embryos cultured in vitro and fused with embryos (Evans et al., Nature, 292:154-156 (1981); Bradley et al., Nature, 309: 255-258 (1984); Gossler et al., Proc. Natl. Acad. Sci. USA, 83: 9065-9069 (1986)); Robertson et al., Nature, 322: 445-448 (1986)). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For a review, see Jaenisch, Science, 240: 1468-1474 (1988).

Conditional, i.e., temporal and spatial, control of gene expression in animals can be achieved using binary transgenic systems, in which gene expression is controlled by the interaction of an effector protein product on a target transgene. These interactions are controlled by crossing animal lines (such as rodent, e.g., mouse lines), or by adding or removing an exogenous inducer, as described in Lewandoski, Nature Reviews Genetics, 2: 743-755 (2001).

Binary transgenic systems fall into two categories. One is based on transcriptional transactivation and is well suited for activating transgenes in gain-of-function experiments. The other is based on site-specific DNA recombination and can be used to activate transgenes or to generate tissue-specific gene knockouts and cell-lineage markers.

The most commonly used transcriptional systems are based on the tetracycline resistance operon of E. coli. The effectors of these systems fall into two categories defined by whether transcription activation occurs upon the administration or depletion of a tetracycline compound (usually doxycycline). The Ga14-based system is a transactivation system that does not require an inducer, but Ga14 transcriptional activation can be controlled by synthetic steroids when a mutated ligand-binding domain is incorporated into a Ga14 chimeric transactivator.

The most widely used site-specific DNA recombination system uses the Cre recombinase from bacteriophage P1, although the Flp recombinase from S. cerevisiae has also been adapted for use in animals such as mice.

By using gene-targeting techniques to produce binary transgene animals with modified endogenous genes that can be acted on by Cre or Flp recombinases expressed under the control of tissue-specific promoters, site-specific recombination may be employed to inactivate endogenous genes in a spatially controlled manner.

Cre/Flp activity can also be controlled temporally by delivering cre/FLP-encoding transgenes in viral vectors, by administering exogenous steroids to the animals that carry a chimeric transgene consisting of the cre gene fused to a mutated ligand-binding domain, or by using transcriptional transactivation to control cre/FLP expression. The irreversibility of site-specific recombination makes this technique uniquely suited for a new type of analysis in which the transient tissue-specific expression of cre/FLP is used to activate permanently a reporter target gene for cell-lineage studies.

Non-human binary transgenic and transgenic animals can be used as tester animals for reagents thought to confer protection from insulin resistance, hyper- or hypoinsulinemia, obesity, or muscle degeneration. In accordance with one facet of this aspect, for example, non-human transgenic animals overexpressing dkk-1 nucleic acid (such as cDNA) in cells (such as muscle cells) can be used to screen candidate drugs (proteins, peptides, polypeptides, small molecules, etc.), for example, for efficacy in increasing glucose clearance from the blood, indicating a treatment for insulin resistance, or in increasing levels of insulin, indicating a treatment for hypoinsulinemia, or in differentiation of muscle cells, indicating a treatment for regeneration of muscles.

In another facet, non-human binary transgenic animals having altered dkk-1 nucleic acid expression can be used to screen candidate drugs as set forth above, such as for their ability to reduce body weight, for example, when exposed to high-fat diets, or adipocytes, indicating a treatment for obesity, or to decrease levels of insulin, indicating a treatment for hyperinsulinemia.

An animal treated with the reagent/drug and having a reduced incidence of the disease, compared to untreated animals bearing the binary or ordinary transgene, would indicate a potential therapeutic intervention for the disease. Assays for these reduced incidence properties are noted above and in the Examples below.

The following Examples are set forth to assist in understanding the invention and should not, of course, be construed as specifically limiting the invention described and claimed herein. Such variations of the inventions that would be within the purview of those in the art, including the substitution of all equivalents now known or later developed, are to be considered to fall within the scope of the invention as hereinafter claimed. The disclosures of all citations herein are incorporated by reference.

### EXAMPLE 1

### Effects of Dkk-1 In Vivo and In Vitro

Materials and Methods

L6 Cell culture

L6 myoblasts were proliferated in growth medium, composed of MEM alpha (Gibco-BRL) with 10% fetal calf serum. Before confluence was reached the cells were dispersed with trypsin and seeded again in fresh growth medium. Myoblast fusion was induced by changing the medium to differentiation medium at confluence (MEM alpha with 2% fetal calf serum). Cells were grown in this medium for 3-9 days and for Dkk-1 treatments longer than 28 hours, dkk-1 (Krupnik et al., supra; WO 99/46281; DNA encoding PRO 1008) was added to this medium. Treatments shorter than 28 hrs were performed in MEM alpha with 0.5% FBS.

Expression of Recombinant Dkk-1

The human homolog of Dkk-1 (hDkk-1) was expressed as a C-terminal 8×His tag fusion (see Krupnik et al., supra; and WO 99/46281, where PRO1008 is Dkk-1) in baculovirus and purified by nickel affinity column chromatography. The identity of purified protein was verified by N-terminal sequence analysis. The purified protein was less than 0.3 EU/ml endotoxin levels.

2-DOG Uptake

Control cells and cells treated with dkk-1 were incubated in Krebs-Ringer phosphate-HEPES buffer (KRHB) (130 mM NaCl, 5 mM KCl, 1.3 mM CaCl₂, 1.3 mM MgSO₄, 10 mM Na2HPO₄, and 25 mM HEPES, pH 7.4) containing 0.5 μCi of 2-deoxy[¹⁴C] glucose in the presence or absence of 0.5 μM insulin for 20 min at 37° C. The cells were washed twice with KRHB, lysed in 100 mM NaOH and the intracellular 2-deoxy[¹⁴C] glucose in the cell lysates was measured by liquid scintillation (LSC).

Quantitation of Gene Expression

Total RNA was isolated using RNeasy Mini Kit (Qiagen) (for cultured cells) or Trizol reagent (Gibco) (for muscle) followed by treatment with DNase I (Amplification Grade, GibcoBRL). Gene expression analysis was performed by Real Time Quantitative-PCR (RTQ-PCR) using an ABI PRISM® 7700 sequencing-detection system (instrument and software supplied by Applied Biosystems, Inc., Foster City, Calif.) as described by Gibson et al., Genome Res., 6: 995-1001 (1996) and Heid et al., Genome Res., 6: 986-994 (1996).

Glycogen Synthesis

Glycogen synthesis was determined as [¹⁴C]glucose incorporation into glycogen. Control L6 cells and cells treated with dkk-1 were incubated for 2 hours in serum-free MEM alpha containing [U-¹⁴C] glucose (5 mM glucose; 1.25 pCi/ml) with or without 0.5 μM insulin. The experiment was terminated by removing the medium and rapidly washing the cells three times with ice-cold PBS, and lysing them with 20% (w/v) KOH, which was neutralized after 1 hour by the addition of 1 M HCl. The lysates were boiled for 5 min, clarified by centrifugation, and the cellular glycogen in the supernatant was precipitated with isopropanol at 0° C. for 2 hours using 1 mg/ml cold glycogen as a carrier. The precipitated glycogen was separated by centrifugation, washed with 70% ethanol, and redissolved in water, and the incorporation of [¹⁴C] glucose into the glycogen was determined by LSC.

Assays for Kinase Activity

Kinases were immunoprecipitated and assayed using reagents from Upstate Biotechnologies, Inc. (Lake Placid, N.Y.) in which the absolute levels of ³²p incorporation into a specific peptide substrate were measured. Specifically, cells were washed with serum-free medium and incubated for 3-5 hr before assay. Cells were stimulated with 30 nM insulin for 30 min, washed in ice-cold PBS followed by lysis in ice-cold solubilization buffer (50 mM Tris-HCl, pH 7.7/0.5% NONIDET P-40™ 4-nonylphenolpolyethyleneglycol low-foam surfactant (Roche Diagnostics GmbH)/2.5 mM EDTA/10 mM NaF/0.2 mM Na₃VO₄/1 mM Na₃MoO₄/1 μg/ml microcystin-LR/0.25 mM phenylmethylsulfonyl fluoride/1 μM pepstatin/0.5 μg/ml leupeptin/10 μg/ml soybean trypsin inhibitor). Antibody (2 μg) against the respective peptide was captured with 40 μl of Protein-G Sepharose beads overnight at 4° C. followed by washing of the beads three times with fresh solubilization buffer. The lysates were clarified by centrifugation (20,000×g, 1 min) and the supernatants were incubated with Protein G-bound antibody at 4° C. for 2 hr with continuous mixing. The beads were washed three times with fresh solubilization buffer containing and once with kinase buffer (20 mM HEPES, pH 7.2/1 mM MgCl₂/1 mM EGTA/1 mM DTT/0.25 mM PMSF/1 mM Na₃VO₄/0.5 μg/ml leupeptin). Beads were resuspended to 30 μl in kinase buffer containing the specific peptide substrate. ATP solution (5 μl) (200 μM ATP/10 μCi ³²P-ATP in kinase buffer) was added followed by incubation for 15 min at 30° C. Reactions were stopped by spotting 20 μl of the reaction volume onto of P81 filter paper, followed by extensive washing with 1% (vol/vol) phosphoric acid and measurement of bound radioactivity by LSC.

For measurement of Akt activity in muscle pieces, freshly isolated muscle pieces were incubated for 30 min at 35 C in KRHB containing 8 mM glucose, 32 mM mannitol and 0.1% BSA that was saturated with O₂/CO₂ (95%/5%) and allowed to recover. The pieces were stimulated with insulin (33 nM and 100 nM) for 10 min, after which the muscle was flash frozen, homogenized in solubilization buffer and clarified by centrifugation. Equal amounts of lysate protein were used for immunoprecipitation of Akt and measurement of Akt activity as described above.

Culture of 3T3/L1 Adipocytes

3T3 μl fibroblasts were grown to confluence and differentiated to adipocytes (Rubin et al., J. Biol. Chem., 253: 7570-7578 (1978)). Differentiated cells were treated with Dkk-1 at 72 hours after the induction of differentiation. For effect of Dkk-1 on 3T3L1 cell differentiation, Dkk-1 was added to the medium at a concentration of 40 nM during the initiation of differentiation and kept throughout the experiment.

Glucose Incorporation into Lipids

Control and treated 3T3 L1 adipocytes were incubated with D-[U-¹⁴C]glucose (0.2 μCi/ml) in serum-free MEM alpha, for 2 hours at 37° C. in the presence or absence of 0.5 μM insulin. The cells were washed twice with ice-cold PBS and lysed in 100 mM NaOH. The lysates were neutralized with 100 mM hydochloric acid and the cellular lipids in the lysates were extracted into n-heptane and the incorporation of [¹⁴C]glucose into the extracted lipid was measured by LSC.

Animals and Diets

All protocols were approved by an Institutional Use and Care Committee. Unless otherwise noted, mice were maintained on standard lab chow in a temperature- and humidity-controlled environment. A 12-hour (6.00 pm/6.00 am) light cycle was used.

Standard mouse chow was PURINA 5010™ brand food (Harlen Teklab, Madison Wis.). The high-fat (58% kJ fat) and low-fat (10.5% kJ fat) isocaloric diets were based on the diets described by Surwit et al., Metabolism 44: 645-651 (1995)) and were purchased from Research Diets (New Brunswick, N.J.).

The human dkk-1 cDNA (Krupnik et al., supra) was ligated 3′ to the pRK splice donor/acceptor site that was preceded by the myosin light-chain promoter (Shani, Nature, 314: 283-286 (1985)). The dkk-1 cDNA was followed by the splice donor/acceptor sites present between the fourth and fifth exons of the human growth hormone gene (Stewart et al., Endocrinology, 130: 405-414 (1992)). The entire expression fragment was purified free from contaminating vector sequences and injected into one-cell mouse eggs derived from FVB×FVB matings. Transgenic mice were identified by PCR analysis of DNA extracted from tail biopsies.

In Vivo Metabolic Measurements and Serum Analysis

Glucose tolerance tests (GTT) were performed by injecting each mouse intraperitoneally with 1.5 mg glucose per gram body weight. Insulin tolerance tests (ITT) were performed by injecting each mouse intravenously with 0.6 U insulin per kg body weight. For both tests, whole blood glucose was measured at the indicated times using a LIFESCAN Fast Take™ glucose meter. Serum levels of insulin and leptin were assayed by ELISA kits (Crystal Chem, Chicago, Ill.). Serum levels of free fatty acids and triglycerides were assayed by NEFA C™ non-esterified fatty acid (Wako Chemicals USB, Inc.) and Sigma Triglyceride, INT™ (Sigma) assay kits, respectively.

Data Analysis

Unless otherwise noted, all data are presented as the means plus and minus the standard deviations. Comparisons between control and treated cells and between transgenic and wild-type mice were made using an unpaired student's t test.

Results

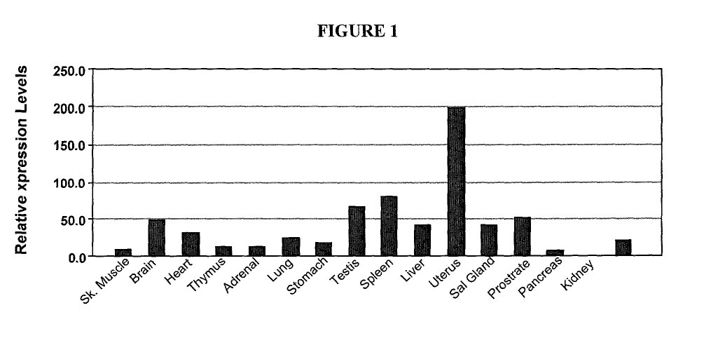

Relative expression levels of dkk-1 in various adult human tissues were determined by Real Time Quantitative PCR (Gibson et al., supra; Heid et al, supra). The results, shown in FIG. 1, indicate that dkk-1 is widely expressed in adult human tissues, and particularly in the spleen, testis, and uterus, and most especially in the uterus.

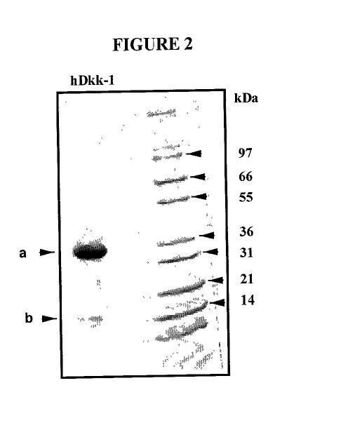

When expressed in baculovirus, the human Dkk-1 protein was clipped internally to give a 16-kDa cleavage product. In the gel shown in FIG. 2, band (a) corresponds to the full-length protein with N-terminal sequence TLNSVLNSNAI (SEQ ID NO:1), with SVLNSNAIKNL (SEQ ID NO:2) corresponding to the signal peptide cleavage site, and band (b) corresponds to the clipped protein with N-terminal sequence SKMYHTKGQE (SEQ ID NO:3).

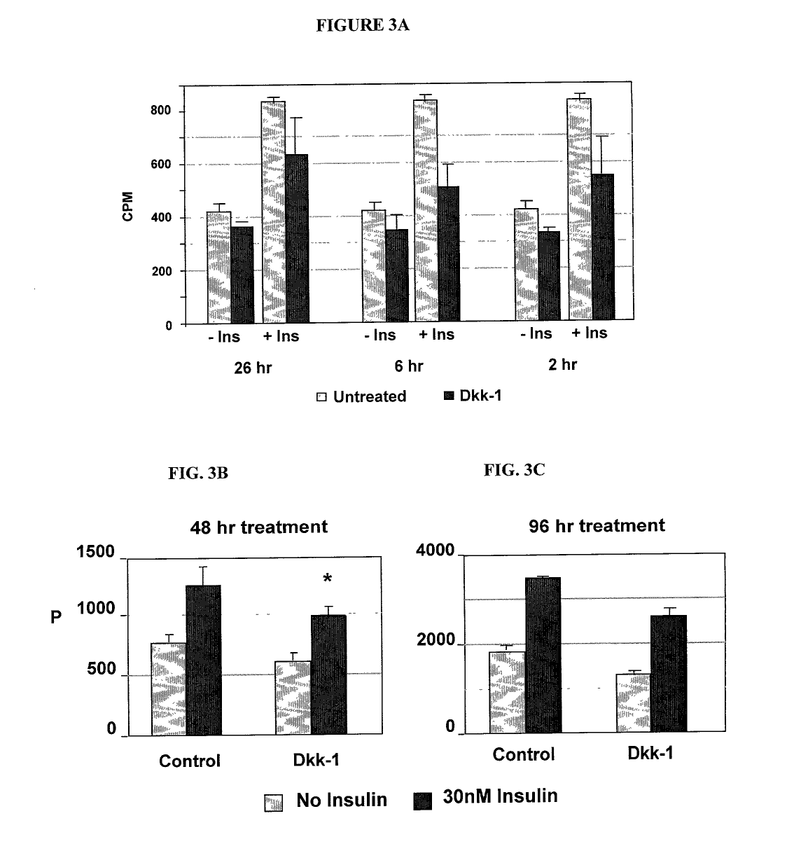

Treatment of L6 muscle cells with Dkk-1 resulted in a reduction of basal and insulin-stimulated glucose uptake in the cells. The effects of Dkk-1 can be seen in as little as 2 hours (FIG. 3A). The effects of short-term treatment are most significant between 2 and 6 hours of treatment. With the long-term treatments (FIGS. 3B and 3C), the decrease in insulin-dependent glucose uptake is more significant at 96 hours (p=0.001), although the effect is seen even at 48 hours (p=0.05).

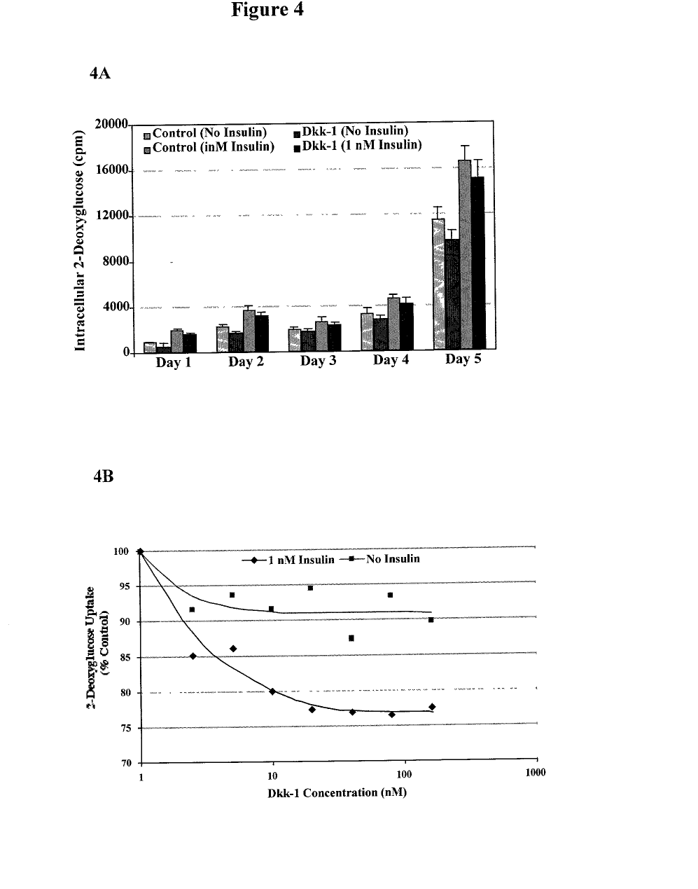

The Dkk-1 effects of glucose uptake are independent of the differentiation state of the cells and can be seen even in cells that are beginning to differentiate to myocytes (FIG. 4A). The effects of Dkk-1 on glucose uptake are dose-dependent. FIG. 4B shows that the decrease in basal and insulin-dependent glucose uptake is seen upon 48-hour treatment with Dkk-1 at concentrations as low as 10 nM.

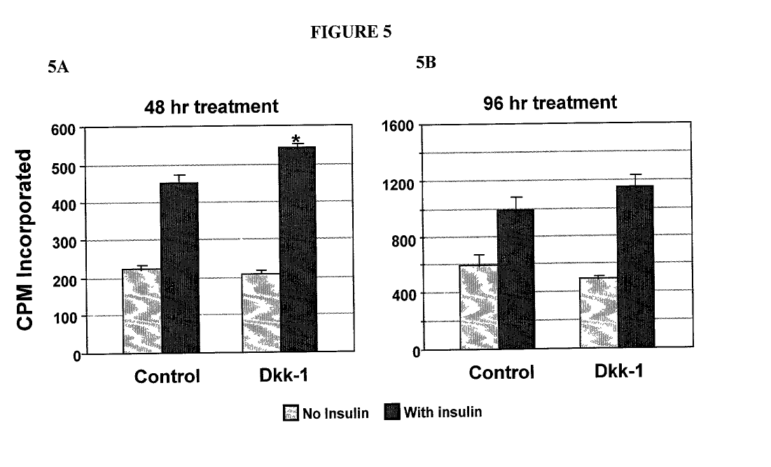

Treatment of L6 muscle cells with Dkk-1 resulted in an increased incorporation of glucose into glycogen. As shown in FIG. 5, the stimulatory effects of Dkk-1 can be seen in 48 hours (p=0.003).

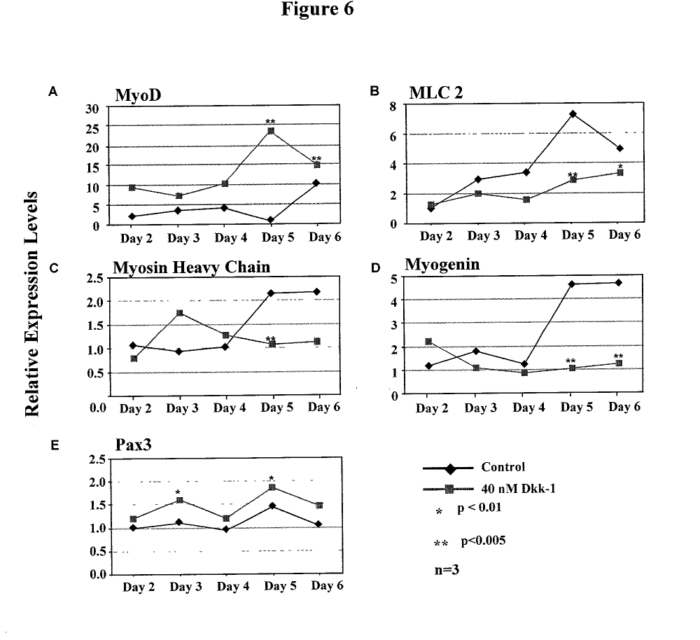

Since the effects of Dkk-1 were observed following long-term treatment, without being limited to any one theory, it is possible that the protein acts by affecting the differentiation of L6 cells. RT-PCR analysis using TAQMAN® Primer and Probe design (Applied Biosystems) was carried out to determine the expression levels of genes involved in myogenesis such as myosin heavy chain (MHC), myosin light chain (MLC), myogenin, Pax3, Myf5, and MyoD in L6 cells treated with Dkk-1. FIG. 6A shows that Dkk-1 treatment resulted in an increase in the levels of MyoD between days 4-6 of differentiation, FIGS. 6B, 6C, and 6D show a decrease in the expression of MLC2, MHC, and myogenin, respectively, on days 4-6 of differentiation, but FIG. 6E shows no significant effect on expression of Pax3. Hence, Dkk-1 regulates myogenesis in L6 cells.

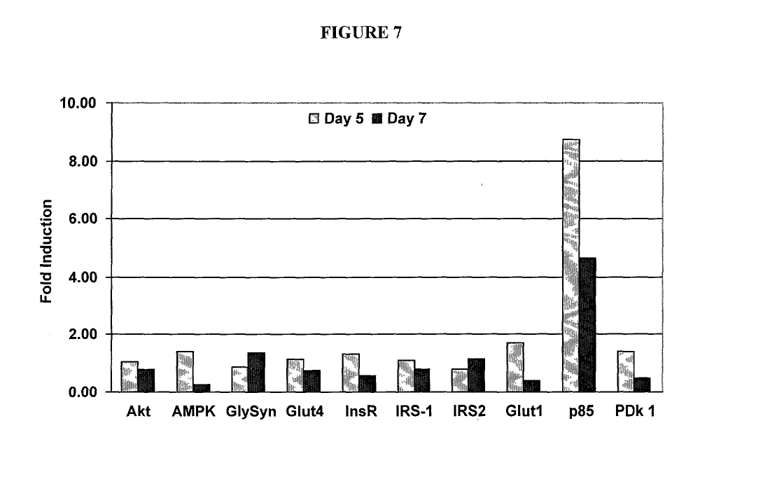

Since Dkk-1 did not significantly affect differentiation of L6 cells, RT-PCR analysis (TAQMAN® Primer and Probe design) was carried out to determine whether Dkk-1 affected the expression levels of genes involved in glucose metabolism. It was found that Dkk-1 regulated the expression of genes in the insulin signaling pathway in L6 muscle cells. In particular, as shown in FIG. 7, Dkk-1 treatment increased the expression of the p85 subunit of phosphoinositide 3-kinase significantly (8.3 fold) following 48-hour treatment, but did not significantly affect expression of other genes tested.

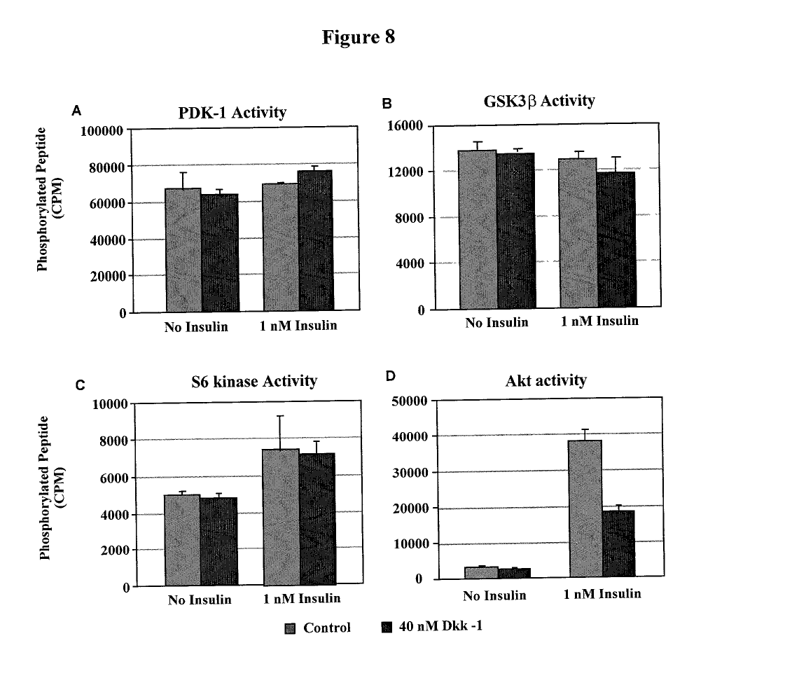

Dkk-1 treatment of L6 muscle cells did not affect the activity of PDK-1 (FIG. 8A), GSK3β (FIG. 8B), or S6 kinase (FIG. 8C), but significantly reduced the level of Akt activity after 48 hours of treatment. Specifically, Dkk-1-treated L6 cells showed a 49% decrease in insulin-stimulated Akt activity (FIG. 8D), which is consistent with the decrease in glucose uptake.

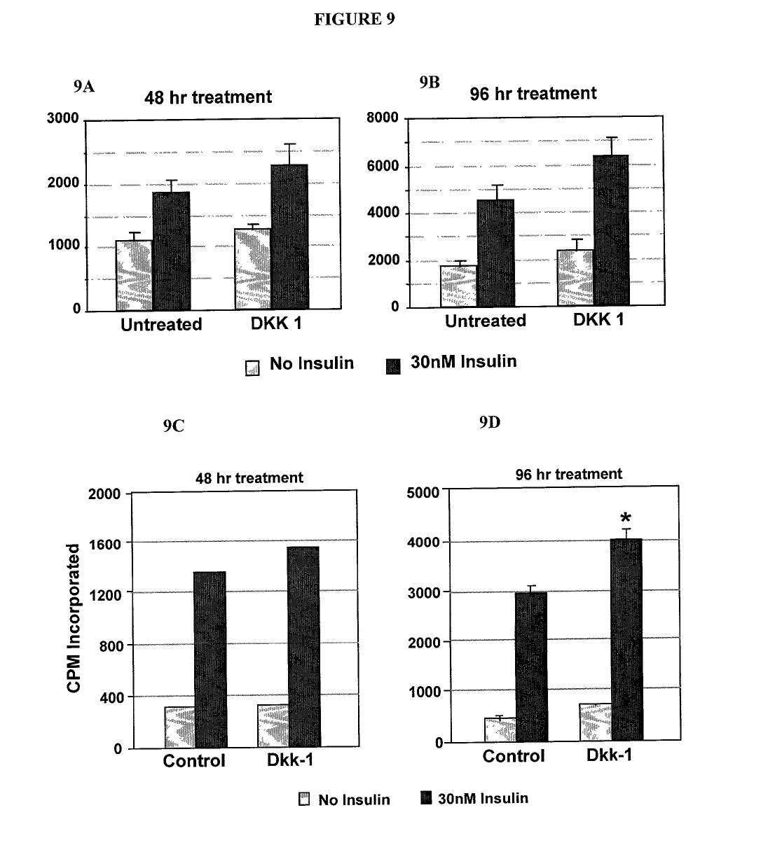

Dkk-1 affected glucose metabolism in adipocytes. Specifically, Dkk-1-treated 3T3 L1 cells showed an increase in levels of basal and insulin-stimulated glucose uptake (FIGS. 9A and 9B) as well as an increased incorporation of glucose into lipids following insulin-stimulation (FIGS. 9C and 9D). The increase in insulin-dependent glucose uptake seen at 48-hour treatment was more pronounced following 96-hour treatment (p=0.04), and a similar observation was seen with the insulin-dependent incorporation of glucose into lipid (p=0.003 after 96 hour treatment).

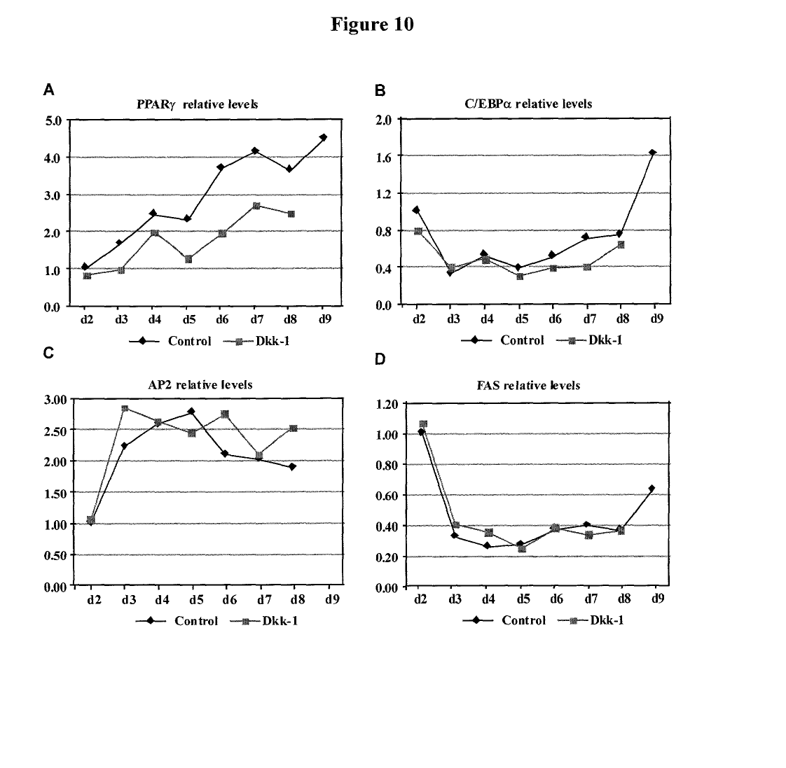

Dkk-1 affected differentiation of adipocytes. Specifically, Dkk-1-treated 3T3 L1 cells showed a decrease in levels of PPARγ and C/EBPα transcripts during differentiation (FIGS. 10A and 10B), although expression of other markers of adipocyte differentiation, such as AP2 and fatty acid synthase (FAS), were not affected (FIGS. 10C and 10D).

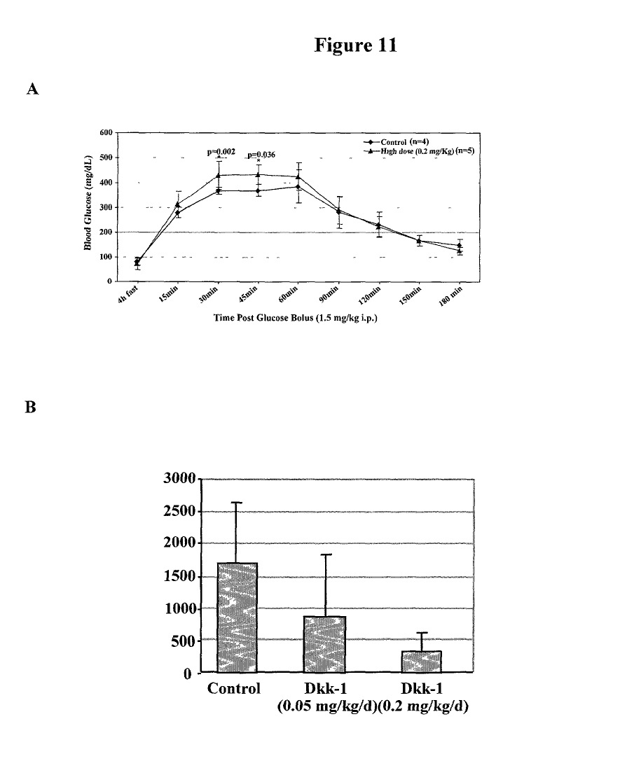

Intravenous injection of recombinant Dkk-1 in mice resulted in impaired glucose tolerance and reduced insulin production. Specifically, to confirm the in vivo effects of Dkk-1 seen in transgenic mice, female FVB mice were injected intravenously with Dkk-1 for 8 days (single daily injection of 0.05 and 0.2 mg/kg/day). The effects of Dkk-1 on glucose tolerance were measured 48 hours and 8 days after the start of injection. Glucose tolerance was unaffected with 48 h of i.v. injection; however, after 8 days of injection animals injected with Dkk-1 at 0.05 or 0.2 mg/kg/day were found to have a reduced rate of glucose clearance from the bloodstream, compared to that seen in saline-injected animals (FIG. 11A). The levels of glucose-induced serum insulin were measured in serum collected 30 min post i.p. glucose injection during the GTT. Animals injected with Dkk-1 had significantly reduced levels of serum insulin compared to that in the control animals, and this reduction was dependent on the dose of Dkk-1 (FIG. 1l B). Insulin tolerance and serum levels of triglycerides, FFA, and leptin were unaffected in Dkk-1-injected animals.

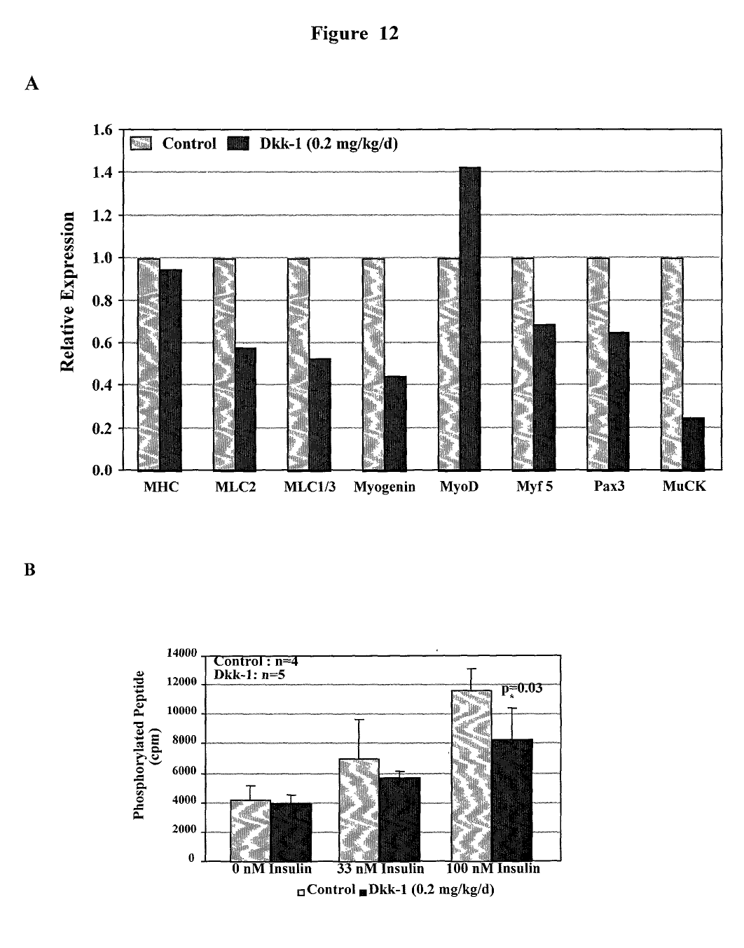

Intravenous injection of recombinant Dkk-1 in mice altered expression of muscle-specific genes and decreased insulin-stimulated Akt activity in muscle in vivo. Specifically, control and Dkk-1-injected animals were fasted for 12-16 hours and sacrificed after 8 days of i.v. injection. Quadriceps muscle was used for extraction of total RNA and RTQ-PCR was used to measure the effects of Dkk-1 on expression of various markers of muscle differentiation such as MyoD, myogenin, MLC2, MLC1/3, myf5, pax3, desmin, and myosin heavy chain. It was observed that Dkk-1-injected animals had decreased expression of MLC2, MLC1/3, myogenin, myf5, Pax3, and muscle creatine kinase (MuCK), but increased expression of MyoD (FIG. 12A), consistent with the effects in L6 cells, suggesting that Dkk-1 affects muscle differentiation in vivo as well, without being limited to any one theory. Expression levels of genes involved in insulin signaling were marginally affected in Dkk-1-injected animals, suggesting that these effects were secondary to effects on muscle differentiation, without being limited to any one theory.

The soleus muscle of control and Dkk-1-injected animals was isolated as described above, and Akt activity was measured in untreated and insulin-treated soleus muscle pieces as described in Oku et al., Am. J. Physiol. Endocrinol. Metab., 280: E816-24 (2001). As shown in FIG. 12B, Dkk-1 treatment resulted in decreased activation of Akt by insulin, consistent with the effects seen in cultured L6 cells.

Overexpression of Dkk-1 in mice affected growth, body composition, and metabolism. Particularly,

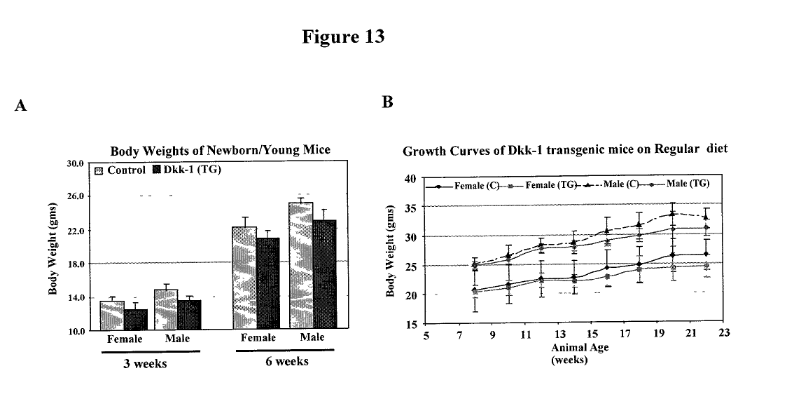

Transgenic FVB mice overexpressing the dkk-1 transgene under control of the MLC promoter were generated (Shani, supra). Body weights of control and transgenic animals were followed over several weeks. As seen in Table 2, transgenic animals on a regular diet had reduced body weights compared to their control littermates. These effects were evident from as early as 10 days of age (FIG. 13A) and could be observed until 22 weeks of age (FIG. 13B).

TABLE 2

| | | | Dkk-1 | Dkk-1 |
|---|---|---|---|---|
| | Control Regular | Control Regular | transgenic | transgenic |
| | diet | diet | Regular diet | Regular diet |
| Physiological Parameter | (males, n = 8) | (females, n = 4) | (males, n = 4) | (females, n = 8) |
| Body Weight at 16 wks of age | 30.6 ± 2.2 | 24.1 ± 3.2 | 28.9 ± 0.9 | 22.7 ± 1.5 |
| (g) | | | | |
| Fasting FFA level | 20.84 ± 3.93 | 15.71 ± 3.11 | 18.26 ± 3.28 | 16.32 ± 4.19 |
| (nMole/5 μl) | | | | |
| Fed FFA level | 10.54 ± 1.85 | 10.93 ± 1.83 | 9.95 ± 0.66 | 10.42 ± 1.86 |
| (nMole/5 μl) | | | | |
| BasalTriglyceride level | 1.17 ± 0.14 | 1.21 ± 0.07 | 1.15 ± 0.08 | 1.13 ± 0.13 |
| (mg/ml) | | | | |
| Triglyceride level (18-hr | 1.96 ± 0.6 | 1.56 ± 0.41 | 1.62 ± 0.36 | 1.57 ± 0.49 |
| fasted) (mg/ml) | | | | |
| Serum insulin (ng/ml) (30 min | 2.55 ± 1.25 | 2.22 ± 9.6 | 1.89 ± 1.56 | 1.47 ± 8.5 |
| post i.p. glucose) | | | | |
| Serum insulin (basal) | 8.7 ± 2.1 | 4.97 ± 2.9 | 6.4 ± 2.1 | 4.7 ± 2.5 |
| (ng/ml) | | | | |
| Serum insulin (18 h fasting) | 1.3 ± 0.3 | 1.68 ± 0.1 | 1.6 ± 0.3 | 1.48 ± 0.3 |
| (ng/ml) | | | | |
| Serum leptin levels (ng/ml) | 16.15 ± 5.0 | 22.0 ± 2.7 | 9.89 ± 5.1 | 11.77 ± 5.7 |
| (fed) | | | | |
| Serum leptin levels (ng/ml) | 4.84 ± 3.2 | 10.07 ± 2.4 | 2.55 ± 2.5 | 4.30 ± 2.6 |
| (20-h fasting) | | | | |

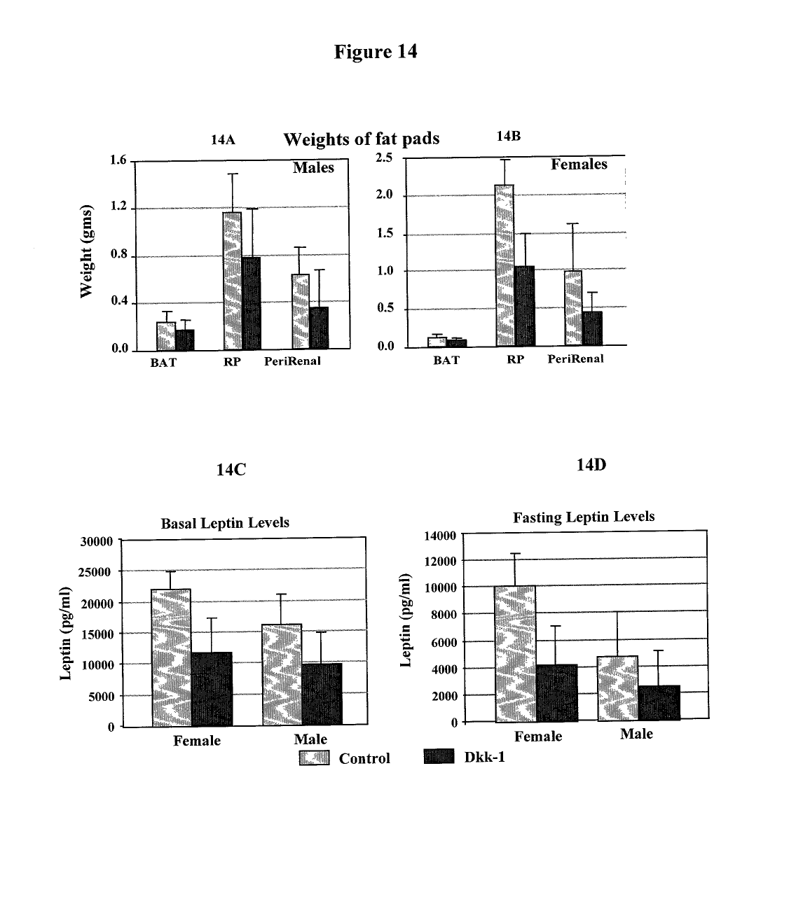

Measurement of weights of various organs (liver, kidney, spleen) and fat pads (brown adipose tissue, retroperitoneal fat, and perirenal fat) revealed that transgenic animals had a proportional reduction in the size of vital organs. However, the weights of fat pads in transgenic animals on a regular or high-fat diet were significantly (40-50%) smaller than in control littermates (FIGS. 14A and 14B). Serum levels of triglycerides, free fatty acids (FFA), and leptin under fasting and fed conditions were measured. Although the levels of triglycerides and free fatty acids were comparable in transgenic and control animals, transgenic animals had almost 50% lower levels of circulating leptin (FIGS. 14C, 14D, Table 2).

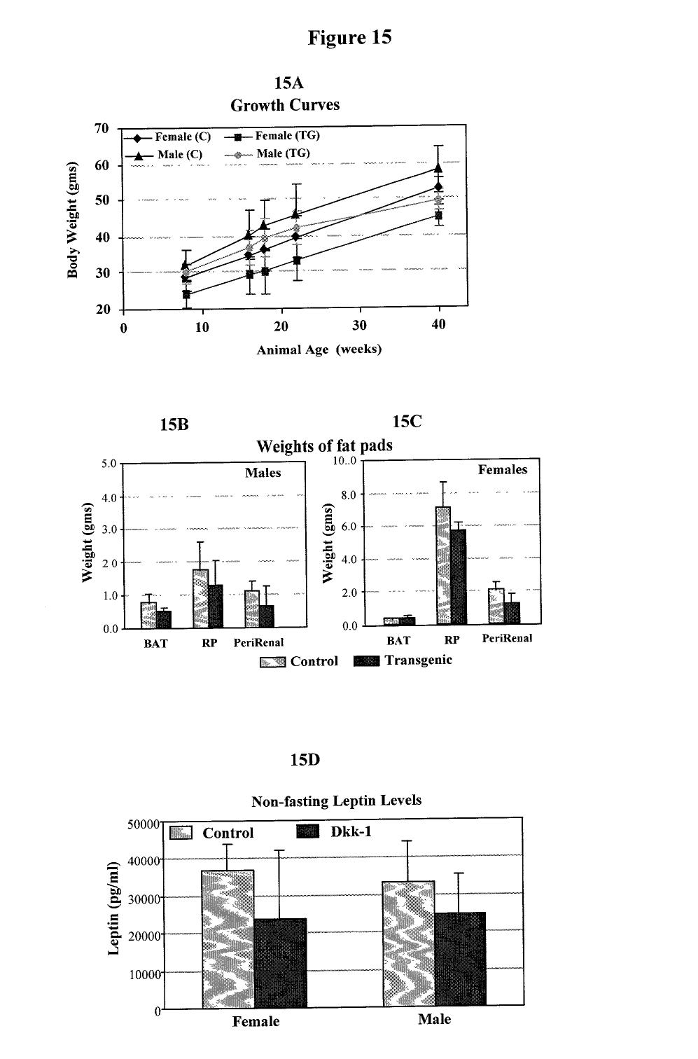

Wnt signaling inhibits adipogenesis. To determine whether Dkk-1 affected body composition, some animals were placed on a high-fat diet for 24 weeks. Dkk-1 transgenic animals on a high-fat diet also showed significantly reduced body weights than their wild-type littermates (FIG. 15A), with comparable reduction in weight of vital organs. Similar to the observations in animals on a regular diet, the fat pads were 40-50% smaller in transgenic animals (FIG. 15B), with comparable reductions in levels of circulating leptin (FIG. 15C). The levels of triglycerides and free fatty acids were comparable in transgenic and control animals (Table 3).

TABLE 3

| | Control | Control | Dkk-1 TG | Dkk-1 TG |
|---|---|---|---|---|
| | High-fat diet | High-fat diet | High-fat diet | High-fat diet |
| Physiological Parameter | (m = 12) | (f = 8) | (m = 6) | (f = 5) |
| Body Weight at 16 wks of age | 40.3 ± 6.6 | 34.7 ± 7.1 | 36.7 ± 4.8 | 29.2 ± 5.1 |
| (g) | | | | |
| Fed FFA level | 9.06 ± 3.3 | 10.92 ± 2.4 | 9.13 ± 2.4 | 10.13 ± 0.68 |
| (nMole/5 μl) | | | | |
| Fed Triglyceride level | 1.08 ± 0.16 | 1.14 ± 0.1 | 1.12 ± 0.12 | 1.19 ± 0.15 |
| (mg/ml) | | | | |
| Serum insulin (30 min. post i.p. | 907.0 ± 645.1 | 327.6 ± 181.2 | 623.0 ± 490.1 | 243.8 ± 103.3 |
| glucose bolus) (pg/ml) | | | | |
| Serum insulin (20-h fasting) | 917.5 ± 726.0 | 714.8 ± 228.4 | 938.0 ± 427.3 | 845.8 ± 606.1 |
| (pg/ml) | | | | |
| Serum leptin levels (ng/ml) | 33.5 ± 10.1 | 36.8 ± 0.6 | 23.6 ± 18.2 | 24.7 ± 10.3 |
| (basal) | | | | |

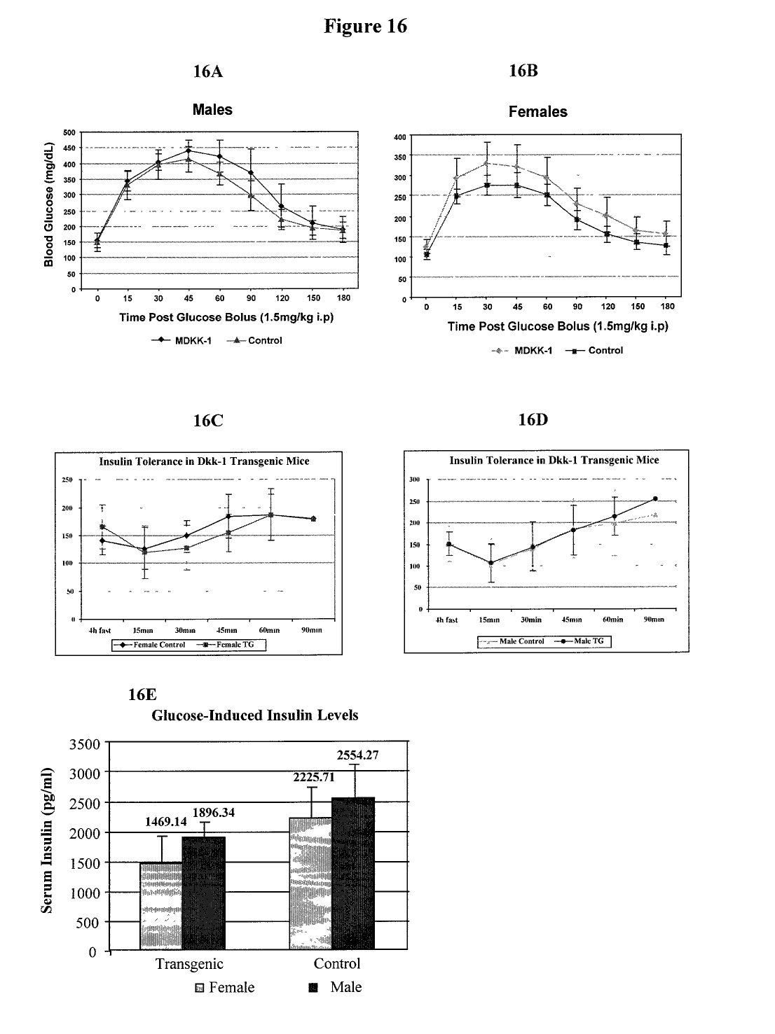

To determine the effects of Dkk-1 on glucose metabolism in vivo, the glucose and insulin tolerance of two independent lines generated from founder transgenic mice transgenic mice was measured. The glucose clearance in the transgenic mice following an intraperitoneal injection of glucose (GTT) was markedly reduced compared to the wild-type littermates in both females and males on a regular diet (FIGS. 16A and 16B), as well as on a high-fat diet. The insulin tolerance was measured in animals on a regular diet and found to be unaffected (FIGS. 16C and 16D). The levels of glucose-induced serum insulin in the transgenic animals 30 min post intraperitoneal glucose bolus, as measured by ELISA, were significantly reduced in transgenic animals compared to levels in the control animals (FIG. 16E).

Discussion

Dkk-1 has distinct effects on glucose uptake in muscle cells in vitro. Dkk-1-treated muscle cells were resistant to insulin treatment, and these effects could be seen in as little as 18 hrs. Insulin resistance, a characteristic of Type 2 diabetes, can be affected by expression levels, phosphorylation, and activity of proteins in the insulin-signaling pathway. Therefore, the effects of Dkk-1 in muscle both in vivo and in vitro were investigated.

The most dramatic effect of Dkk-1 in L6 muscle cells was the 50% reduction in the insulin-stimulated activation of Akt, a key kinase in the insulin-signaling pathway. Transgenic animals overexpressing Dkk-1 in muscle had a reduced glucose clearance from the serum, although their insulin tolerance was unaltered. These animals also demonstrated growth retardation and had proportionally smaller lean and fat mass and vital organs compared to their wild-type littermates. The effects of Dkk-1 on glucose clearance and on insulin-stimulated activation of Akt in muscle could be observed in animals following i.v. injection of Dkk-1 for 8 days. These animals also had reduced levels of serum insulin, although no effects were seen in the serum insulin levels in transgenic mice. Dkk-1 reduced the basal and insulin-stimulated glucose uptake in L6 cells through inhibition of Akt, a key intermediate in the insulin-signaling pathway. These effects of Dkk-1 were seen only after 18 hrs of exposure to Dkk-1.

Dkk-1 significantly affected muscle cell differentiation in vitro and in vivo, showing that an antagonist thereof would be useful in regenerating and repairing muscle.

Animals expressing the dkk-1 transgene had a reduced body size with a proportional decrease in the weight of various organs. Without being limited to any one theory, these effects of Dkk-1 are likely to be mediated through the reduction in insulin (and likely IGF-1)-stimulated Akt activity. Direct evidence for this comes from studies in mice in which the gene for Akt1 has been disrupted (Chen et al., Genes and Development, 15: 2203-2208 (2001)). These animals are smaller in size and show reduced body weight at birth and decreased growth rates, although their glucose metabolism is not affected. Additionally, Akt mediates signaling between the growth hormone receptor and the nucleus (Piwien-Pilipuk et al., J. Biol. Chem., 276: 19664-19671 (2001)). Alternatively, without limitation to any one theory, the reduced growth rate in dkk-1 transgenic animals could be a secondary effect of the reduced glucose uptake and consequent alteration in nutrient availability and metabolic rate in these animals. Akt regulates muscle hypertrophy and prevents atrophy (Bodine et al, Nature Cell Biology, 3: 1014-1019 (2001); Rommel et al., Nature Cell Biology, 3: 1009-1013 (2001)), and it is possible, without being limited to any one theory, that the Dkk-1 effects on body size are mediated through Akt-regulated muscle differentiation and/or regeneration.

Dkk-1 transgenic mice have reduced fat pads, suggesting that Dkk-1 affects adipocyte differentiation. Without being limited to any one theory, this may be mediated in part through inhibition of Akt, a known regulator of adipogenesis (Magun et al., Endocrinology, 137: 3590-3593 (1996)).

Primary 3T3L1 preadipocytes were stimulated to differentiate in the presence or absence of Dkk-1, cells were collected at different days after the start of differentiation, and the transcripts analysed for expression levels of markers of adipocyte differentiation such as AP2, PPARγ, CEBPα, and FAS. Dkk-1 treatment did not alter levels of FAS and AP2; however, PPARγ levels were about 2-fold reduced in Dkk-1-treated cells and C/EBPα levels about 1-fold reduced in Dkk-1-treated cells from day 5 to day 8 of differentiation.

PPARγ is a key regulator of adipocyte formation (Hu et al., Proc. Natl. Acad. Sci. USA, 92: 98569860 (1995)); Hallakou et al., Diabetes, 46: 1393-99 (1997)), and a mutation that results in a receptor with increased transcriptional activity has been identified in severely obese patients (Ristow et al., N. Engl. J. Med., 339: 953-959 (1998)). In addition, PPARγ may also play a key role in regulation of insulin sensitivity in muscle. The expression of PPARγ is altered in skeletal muscle of Type 2 diabetics (Lovisacach et al., Diabetologia, 43: 304-311 (2000)) and mutations that impair its transcriptional activity have been identified in individuals with severe insulin resistance and Type 2 diabetes (Barroso et al., Nature, 402: 880-883 (1999)). However, the most compelling evidence for the role of PPARγ in Type 2 diabetes comes from the use of the thiazolidinedione (TZD) class of drugs (glitazones) that are approved for the treatment of human Type 2 diabetes (rosiglitazone/Avandia and pioglitazone/Actos). These drugs are selective PPARγ agonists (Forman et al., Cell, 83: 803-812 (1995)) that ameliorate insulin resistance and lower glucose levels without stimulating insulin secretion by increasing glucose utilization in skeletal muscle through a variety of mechanisms (reviewed in Olefsky and Saltiel, Trends Endo. and Metabolism, 11: 362-367 (2000); Willson et al., Annu. Rev. Biochem. 70:341-67 (2001)).

Adipocyte differentiation is stimulated by constitutively active Akt (Magun et al., Endocrinology, 137: 3590-3593 (1996)). Serum leptin levels are dependent on adipose tissue mass and are up-regulated by Akt (Barthel et al., Endocrinology, 138: 3559-3562 (1997)). The reduced levels of circulating leptin in dkk-1 transgenic animals could be a direct effect of decreased adipose mass and/or decreased Akt activity in adipose tissue, without being limited to any one theory.

The most well studied function of Akt is its role in glucose metabolism. In response to insulin, Akt regulates IRS-1 function (Paz et al., J. Biol. Chem., 274: 28816-28822 (1999)) and phosphorylation and activity of GSK3β (Ross et al., Mol. Cell. Biol., 19: 8433-8441 (1999); Summers et al., J. Biol. Chem., 274: 17934-17940 (1999)), phosphorylates components of GLUT-4 vesicles, and regulates GLUT4 translocation to the cell surface (Kupriyanova and Kandror, J. Biol. Chem., 274: 1458-1464 (1999); Wang et al., Mol. Cell. Biol., 19: 4008-4018 (1999)). Decreased phosphorylation of Akt (Krook et al., 1998, supra) has been observed in skeletal muscle of some Type 2 diabetic subjects, and in obese animals (Carvalho et al., Diabetologia, 43: 1107-1115 (2000); Kim et al., supra; Shao et al., J. Endocrinol., 167: 107-115 (2000)). In addition, mice in which the Akt2 gene is disrupted have the Type 2 diabetic phenotype (Cho et al., Science, 292: 1728-1731 (2000)). Further, Akt activity in vivo is affected by several conditions that result in altered glucose metabolism such as hyperglycemia (Kurowski et al., Diabetes, 48: 658-663 (1999); Nawano et al., Biochem. Biophys. Res. Commun., 266: 252-256 (1999); Oku et al., supra), muscle damage (Del Aguila et al., Am. J. Physiol. Endocrinol. Metab., 279: E206-212 (2000), glycogen content (Derave et al., Am. J. Physiol. Endocrinol. Metab., 279: E947-955 (2000)), and high-fat diet (Tremblay et al., Diabetes, 50: 1901-1910 (2001)).

In addition to its role in differentiation and glucose metabolism, Akt is believed to play a key role in proliferation (Holst et al., Biochem. Biophys. Res. Commun., 250: 181-186 (1998); Trumper et al., Ann. N.Y. Acad. Sci., 921: 242-250 (2000); Tuttle et al., Nat. Med., 7: 1133-1137 (2001); Bernal-Mizrachi et al., J. Clin. Invest., 108: 1631-1638 (2001)) and survival (Aikin et al., Biochem. Biophys. Res. Commun., 277: 455-461 (2000)) of insulin-secreting pancreatic β-cells. Further, impairment of early steps in insulin signaling may decrease beta-cell survival and cause resistance to antiapoptotic effects of insulin by affecting the PI3-kinase/Akt survival pathway (Federici et al., Faseb J., 15: 22-24 (2001)). Overexpression of Akt1 in β-cells results in a significant increase in both β-cell size and total islet mass, and this is accompanied by increased levels of serum insulin, improved glucose tolerance, and resistance to streptozotocin-induced diabetes (Tuttle et al., supra; Bernal-Mizrachi et al., supra).

A significant reduction in the levels of secreted insulin was observed herein following 8 days of Dkk-1 injection, and smaller effects in transgenic animals overexpressing dkk-1 in the muscle. Without being limited to any one theory, the stronger effects in injected animals could be a result of direct effects on pancreatic β-cell survival via inhibition of Akt, while in transgenic animals there may be smaller differences in insulin levels either due to compensatory mechanisms or due to a more localized effect of Dkk-1 in the muscle. Since Akt is known to stimulate islet cell proliferation and insulin production, and since the data herein show for the first time that Dkk-1-injected and transgenic mice have lower insulin levels, an antagonist to Dkk-1 is now found useful in treating hypoinsulinemia, and conversely, Dkk-1 itself is found useful in treating hyperinsulinemia.

Conclusion

Dkk-1 affected glucose metabolism in L6 muscle cells as well as in transgenic mice overexpressing the protein in muscle. Treatment of muscle cells with Dkk-1 resulted in a decrease in the basal and insulin-stimulated glucose uptake. This effect was observed following both short-term and long-term treatment, suggesting, without being limited to any one theory, that Dkk-1 may affect both the activity as well as the expression levels of proteins in the insulin signaling pathway. Consistent with this observation, transgenic mice overexpressing the protein had decreased glucose tolerance, although the levels of serum insulin were not affected. Further, Dkk-1-injected and transgenic animals had lower insulin levels. Dkk-1 also promoted muscle cell differentiation. Finally, Dkk-1 appears to reduce body weight and fat pads. The above observations demonstrate that Dkk-1 induces muscle degeneration, insulin resistance, which is a key feature of most forms of NIDDM, and hypoinsulinemia, and promotes weight loss or reduction in fat tissue and cells. Hence, an antagonist to Dkk-1 would be useful in treating insulin resistance, hypoinsulinemia, and muscle degeneration, and Dkk-1 is useful in treating obesity and hyperinsulinemia, as well as being useful as a diagnostic marker in assays for such conditions. Also, an antagonist to Dkk-1 is expected to inhibit the progression of the diabetes phenotype in transgenic animal models disclosed in U.S. Pat. No. 6,187,991.

### EXAMPLE 2

### Development of Anti-Dkk-1 Monoclonal Antibodies

Five female Balb/c mice (Charles River Laboratories, Wilmington, Del.) were hyperimmunized with purified recombinant polyhistidine-tagged (HIS8) human Dkk-1 expressed in baculovirus (WO 99/46281) and diluted in Ribi adjuvant (Ribi Immunochem Research, Inc., Hamilton, Mo.). The animals were immunized twice per week, with 50 μl used for each animal, administered via footpad. After five injections, B-cells from the lymph nodes of the five mice, demonstrating high anti-Dkk-1 antibody titers, were fused with mouse myeloma cells (X63.Ag8.653; American Type Culture Collection, Manassas, Va.) using the protocols described in Kohler and Milstein, supra, and Hongo et al., Hybridoma, 14: 253-260 (1995). After 10-14 days, the supernatants were harvested and screened for antibody production by direct ELISA. Seven positive clones, showing the highest immunobinding after the second round of subcloning by limiting dilution, which were deposited with the ATCC as noted below, were injected into PRISTANE™ 2,6,10,14-tetramethylpentacane (Aldrich Chemical Co.)-primed mice (Freund and Blair, J. Immunol., 129: 2826-2830 (1982)) for in vivo production of MAb. The ascites fluids were pooled and purified by Protein A affinity chromatography (PHARMACIA™ fast protein liquid chromatography [FPLC]; Pharmacia and Upjohn) as described by Hongo et al., supra. The purified antibody preparations were sterile filtered (0.2-μm pore size; Nalgene, Rochester N.Y.) and stored at 4° C. in phosphate-buffered saline (PBS).

All the seven antibody preparations bound Dkk-1 in Western immunoblots.

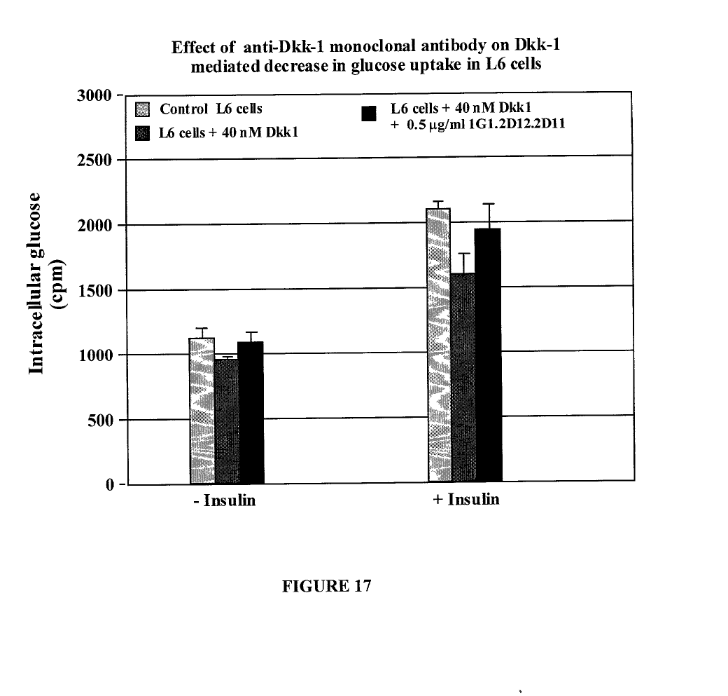

L6 cells were differentiated and treated for 48 hours in the absence of Dkk-1 (control) or in the presence of 40 nM Dkk-1 (plus or minus anti-Dkk-1 antibody 1G1.2D12.2D11 (ATCC No. PTA-3086) in an amount of 0.5 μg/mL). Basal and insulin-stimulated glucose uptake in the L6 cells was measured as described in Example 1. FIG. 17 shows that in both the absence and presence of insulin, the monoclonal antibody neutralized the Dkk-1-mediated decrease in glucose uptake in the L6 cells.

Deposit of Material

The following materials have been deposited with the American Type Culture Collection, 10801 University Blvd., Manassas, Va. 20110-2209, USA (ATCC):

| Designation | ATCC Dep. No. | Deposit Date |
|---|---|---|
| DKK1.MAB3139.8C11.2G11.1D1 | PTA-3084 | Feb. 21, 2001 |
| DKK1.MAB3143.4C7.2H10.2G1 | PTA-3085 | Feb. 21, 2001 |
| DKK1.MAB3142.1G1.2D12.2D11 | PTA-3086 | Feb. 21, 2001 |
| DKK1.MAB3141.5B12.2C5.2A5 | PTA-3087 | Feb. 21, 2001 |
| DKK1.MAB3138.7C11.2H6.2A8 | PTA-3088 | Feb. 21, 2001 |
| DKK1.MAB3140.7B2.2A6.2H4 | PTA-3089 | Feb. 21, 2001 |
| DKK1.MAB3144.5A2.2A8.1C3 | PTA-3097 | Feb. 21, 2001 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC section 122 and the Commissioner's rules pursuant thereto (including 37 CFR section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited materials is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the constructs deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

The principles, preferred embodiments and modes of operation of the present invention have been described in the foregoing specification. The invention that is intended to be protected herein, however, is not to be construed as limited to the particular forms disclosed, since they are to be regarded as illustrative rather than restrictive. Variations and changes may be made by those skilled in the art without departing from the spirit of the invention.

What is claimed is:

1. A method of treating insulin resistance or hypoinsulinemia in mammals comprising administering to a mammal in need thereof an effective amount of an antagonist to Dickkopf-1 (Dkk-1).

2. The method of claim 1 wherein the mammal has non-insulin dependent diabetes mellitus (NIDDM).

3. The method of claim 1 wherein the mammal is human and the antagonist is to human Dkk-1.

4. The method of claim 1 wherein the antagonist is an antibody that binds Dkk-1.

5. The method of claim 4 wherein the antibody is a monoclonal antibody.

6. The method of claim 5 wherein the antibody is prepared from a hybridoma having ATCC Dep. No. PTA-3086.

7. The method of claim 1 wherein the administration is systemic.

8. The method of claim 1 wherein insulin resistance is treated, further comprising administering an effective amount of an insulin-resistance-treating agent to the mammal.

9. The method of claim 1 wherein hypoinsulinemia is treated, further comprising administering an effective amount of insulin to the mammal.

10. A method for detecting the presence or onset of insulin resistance or hypoinsulinemia in a mammal comprising the steps of:
(a) measuring the amount of Dickkopf-1 (Dkk-1) in a sample from said mammal; and
(b) comparing the amount determined in step (a) to an amount of Dkk-1 present in a standard sample, an increased level in the amount of Dkk-1 in step (a) being indicative of insulin resistance or hypoinsulinemia.

11. The method of claim 10 wherein the measuring is carried out using an anti-Dkk-1 antibody in an immunoassay.

12. The method of claim 11 wherein the anti-Dkk-1 antibody comprises a label.

13. The method of claim 12 wherein the label is selected from the group consisting of a fluorescent label, a radioactive label, and an enzyme label.

14. The method of claim 11, wherein the immunoassay is selected from the group consisting of a radioimmunoassay, an enzyme immunoassay, an enzyme-linked immunosorbent assay, a sandwich immunoassay, a precipitation assay, an immunoradioactive assay, a fluoresence immunoassay, a protein A immunoassay, and an immunoelectrophoresis assay.

15. The method of claim 10 wherein the insulin resistance is non-insulin dependent diabetes mellitus.

16. The method of claim 10 wherein the mammal is human and human Dkk-1 is being measured.

17. A kit for treating insulin resistance or hypoinsulinemia, said kit comprising:
(a) a container comprising an antagonist to Dkk-1; and
(b) instructions for using the antagonist to treat insulin resistance or hypoinsulinemia.

18. The kit of claim 17 wherein the antagonist is an antibody that binds Dkk-1.

19. The kit of claim 18 wherein the antibody is a monoclonal antibody.

20. The kit of claim 18 wherein the antibody binds human Dkk-1.

21. The kit of claim 17 for treating non-insulin dependent diabetes.

22. The kit of claim 17 further comprising a container comprising an insulin-resistance-treating agent if insulin resistance is treated or insulin if hypoinsulinemia is treated.

23. A hybridoma selected from the group consisting of ATCC Dep. No. PTA-3084, PTA-3085, PTA-3086, PTA-3087, PTA-3088, PTA-3089, and PTA-3097.

24. The hybridoma of claim 23 that is ATCC Dep. No. PTA-3086.

25. An antibody prepared from the hybridoma of claim 23.

26. A method of treating obesity or hyperinsulinemia in mammals comprising administering to a mammal in need thereof an effective amount of Dickkopf-1 (Dkk-1).

27. The method of claim 26 wherein the mammal is human and the Dkk-1 is human Dkk-1.

28. The method of claim 26 wherein the administration is systemic.

29. The method of claim 26 further comprising administering an effective amount of weight-loss agent.

30. A method for detecting the presence or onset of obesity or hyperinsulinemia in a mammal comprising the steps of:
(a) measuring the amount of Dickkopf-1 (Dkk-1) in a sample from said mammal; and
(b) comparing the amount determined in step (a) to an amount of Dkk-1 present in a standard sample, a decreased level in the amount of Dkk-1 in step (a) being indicative of obesity or hyperinsulinemia.

31. The method of claim 30 wherein the measuring is carried out using an anti-Dkk-1 antibody in an immunoassay.

32. The method of claim 31 wherein the anti-Dkk-1 antibody comprises a label.

33. The method of claim 32 wherein the label is selected from the group consisting of a fluorescent label, a radioactive label, and an enzyme label.

34. The method of claim 31, wherein the immunoassay is selected from the group consisting of a radioimmunoassay, an enzyme immunoassay, an enzyme-linked immunosorbent assay, a sandwich immunoassay, a precipitation assay, an immunoradioactive assay, a fluoresence immunoassay, a protein A immunoassay, and an immunoelectrophoresis assay.

35. The method of claim 30 wherein the mammal is human and human Dkk-1 is being measured.

36. A kit for treating obesity or hyperinsulinemia, said kit comprising:
(a) a container comprising Dkk-1; and
(b) instructions for using the Dkk-1 to treat obesity or hyperinsulinemia.

37. The kit of claim 36 wherein the Dkk-1 is human Dkk-1.

38. The kit of claim 36 further comprising a container comprising a weight-loss agent if obesity is being treated or comprising diazoxide if hyperinsulinemia is being treated.

39. A diagnostic kit for detecting the presence or onset of insulin resistance, hyperinsulinemia, hypoinsulinemia, or obesity, said kit comprising:
(a) a container comprising an antibody that binds Dickkopf-I (Dkk-1);
(b) a container comprising a standard sample containing Dkk-1; and
(c) instructions for using the antibody and standard sample to detect insulin resistance, hyperinsulinemia, hypoinsulinemia, or obesity, wherein either the antibody that binds Dkk-1 is detectably labeled or the kit further comprises another container comprising a second antibody that is detectably labeled and binds to the Dkk-1 or to the antibody that binds Dkk-1.

40. The kit of claim 39 wherein the antibody that binds Dkk-1 is a monoclonal antibody.

41. The kit of claim 39 wherein the Dkk-1 is human Dkk-1 and the kit is for detecting non-insulin dependent diabetes or obesity.

42. A method for repairing or regenerating muscle in a mammal comprising administering to the mammal an effective amount of an antagonist to Dkk-1.

43. The method of claim 42 wherein the antagonist is an antibody that binds Dkk-1.

44. The method of claim 43 wherein the mammal is human and the antibody binds human Dkk-1.

45. The method of claim 42 wherein the antibody is a monoclonal antibody.

46. A kit for repairing or regeneration muscle, said kit comprising:
(a) a container comprising an antagonist to Dkk-1; and
(b) instructions for using the antagonist to repair or regenerate muscle in a mammal.

47. A monoclonal antibody preparation prepared by hyperimmunizing mice with tagged Dkk-1 diluted in an adjuvant, fusing B-cells from the mice having anti-Dkk-1 antibody titers with mouse myeloma cells and obtaining supernatants, harvesting the supernatants, screening the harvested supernatants for antibody production, injecting positive clones showing the highest immunobinding after a second round of subcloning into primed mice for in vivo production of monoclonal antibodies, pooling ascites fluids from the mice, and purifying the ascites fluid pool to produce the antibody preparation.

48. A method of evaluating the effect of a candidate pharmaceutical drug on insulin resistance, hypoinsulinemia, or muscle repair comprising administering said drug to a non-human transgenic animal that overexpresses dkk-1 nucleic acid and determining the effect of the drug on glucose clearance from the blood of said animal, on circulating insulin levels in said animal, or on muscle differentiation, respectively.

49. A method of evaluating the effect of a candidate pharmaceutical drug on obesity or hyperinsulinemia comprising administering said drug to a non-human binary transgenic animal that expresses dkk-1 nucleic acid and determining the effect of the drug on an obesity-determining property or on the level of insulin in said animal.

50. A non-human transgenic animal that overexpresses dkk-1 nucleic acid.

51. The animal of claim 50 that is a rodent.

52. The animal of claim 50 that is a mouse.
